(19) United States
(12) Patent Application Publication
(10) Pub. No.: US 2021/0386580 A1
(43) Pub. Date: Dec. 16, 2021

(54) SYSTEMS AND METHODS FOR GENERATING SLURRY

(71) Applicant: MIRAKI INNOVATION THINK TANK LLC, Cambridge, MA (US)

(72) Inventors: Christopher VELIS, Lexington, MA (US); Tarik S. CHAUDHRY, Boston, MA (US); Karen MILLER, South Dartmouth, MA (US); William Roger MAINWARING-BURTON, Cambridge, MA (US); Bradley Leo GUERTIN, Roseville, MN (US); Avi Aaron KURLANTZICK, Dedham, MA (US); Andrew Arthur DAVENPORT, Englewood, CO (US); Nicholas Robert TOSTA, Boston, MA (US); Bryan Ellis WAGENKNECHT, West Roxbury, MA (US); Rainuka GUPTA, Newton, MA (US)

(73) Assignee: MIRAKI INNOVATION THINK TANK LLC, Cambridge, MA (US)

(21) Appl. No.: 17/283,651

(22) PCT Filed: Oct. 10, 2019
(86) PCT No.: PCT/US2019/055633

§ 371 Date: Apr. 8, 2021

Related U.S. Application Data
(60) Provisional application No. 62/743,830, filed on Oct. 10, 2018.

(51) Int. Cl. A61F 7/00

(52) U.S. Cl. CPC A61F 7/0085; A61F 2007/126

(57) ABSTRACT

Methods and systems for generating sterile slurry are provided. The slurry may be injected into subcutaneous fat of a subject to facilitate weight reduction or improve cosmetic appearances. Systems for generating a slurry comprise a repository for receiving a solution, a generator for generating the slurry from the solution, and a port for transferring the slurry from the system. Methods for generating a slurry comprise receiving a solution in a slurry generator, and generating slurry from the solution, wherein the slurry comprises ice particles capable of flowing through a cannula. Systems include continuous flow systems, agitated systems, and hybrid continuous flow and agitation systems.

### TECHNICAL FIELD

The disclosure relates to systems and methods for generating slurry.

### BACKGROUND

Various medical and cosmetic benefits may be achieved through application of cold devices to the skin. Similarly, a cold composition may be topically applied to stimulate thermogenesis in certain tissues, leading to increased metabolic activity and reduction of fatty tissue. Many such medical and cosmetic benefits may be better attained by depositing the cold composition closer to the site of the tissue or the afflicted portion of the tissue. However, available methods of generating or applying a cold composition to a particular tissue often are painful, have long treatment times, require a visit to a healthcare facility, and require an extensive recovery period. Perhaps most importantly, available cold compositions suffer from limited effectiveness due to changes in the composition when administered as compared to when formulated.

In some cases, a cold composition may be too cold when administered, increasing the likelihood of harmful tissue damage. In other cases, the cold composition may heat up prior to administration, causing degradation and resulting in uncontrolled variation decreasing the effectiveness of the treatment. In other cases, ice is required at the point of care to formulate the cold slurry, which makes it undesirable in a practice setting. In any event, available methods for generating and delivering a cold composition to achieve various medical and cosmetic benefits are unreliable. This is unsatisfactory to many medical practitioners and patients, deterring them from realizing the various health improvements a cold composition may provide.

### SUMMARY

The invention provides systems and methods for generating sterile slurry. The slurry of the present invention can be used in selective injection cryolipolysis for fat removal, selective targeting of non-adipocyte, lipid rich tissue, and connective tissue remodeling, while avoiding non-specific hypertonic injury to tissue. Systems of the invention comprise a continuous flow system, an agitation system, and a hybrid continuous flow and agitation system. The resultant slurry is safe for injection and use in humans because the slurry includes biocompatible ingredients, such as water, ice, and other biocompatible additives.

Systems of the invention generate and maintain slurry in a manner that is consistent in temperature, ice particle size, and ice coefficient, among other properties. For example, in a treatment that involves multiple injections in different treatment areas or multiple injections in a single treatment area, the variation between the first and last injections, subsequent injections in a series, and injections in subsequent treatment events is minimized. Further, the slurry may be tailored to lessen adverse effects, such as pain and redness, associated with injection. This improves the medical and cosmetic benefits potentially realized by injection of the slurry.

Certain embodiments of the invention are directed to systems for generating a slurry. The systems comprise a repository for receiving a solution, a generator for generating the slurry from the solution, and a port for transferring the slurry from the system.

In some embodiments, the system is a continuous flow system. In some embodiments, the system is an agitated system. In some embodiments, the agitated system is an agitated syringe. In some embodiments, the system is a hybrid continuous flow and agitation system.

The solution may comprise liquid water and one or more additives. The one or more additives may affect tonicity and/or flowability of the slurry. In some embodiments, the one or more additives comprise one or more of a salt, a sugar and a thickener. In some embodiments, Additives can include any substance on the FDA GRAS list for example, sodium chloride, glycerol, sodium carboxymethylcellulose (CMC), dextrose, xanthan gum, glycerin, polyethylene glycol, cellulose, polyvinyl alcohol, polyvinylpyrrolidone, guar gum, locust bean gum, carrageenan, alginic acid, gelatin, acacia, and carbopol.

The slurry may comprise ice particles capable of flowing through a cannula. For example, the ice coefficient (defined as the percentage of ice by weight in the slurry), ice shape, and ice quality generated is suitable for injection through the cannula. In some embodiments, the cannula is a needle, for example, a needle having a gauge size of about 8 G to about 25 G.

The slurry may have an ice coefficient of about 2% to about 70%. The slurry temperature can range from about −25° C. to about 10° C. In some embodiments, the temperature is from about −6° C. to about 0° C. The slurry may have a pH from about 4.5 to about 9. The ice particles in the slurry may have a particle size of less than about 1 mm. The slurry may have an osmolality of less than about 2,200 milli-Osmoles/kilogram. In some embodiments, the slurry has an osmolality of less than about 600 milli-Osmoles/kilogram.

The slurry is configured to be introduced to a patient. The slurry is suitable for administration via injection into subcutaneous fat of the patient. Because the slurry is configured to be injected to a subject such as a human, sterility is important. In some embodiments, the system comprises a port for aseptically transferring the slurry to a delivery device. In some embodiments, the delivery device is disposable. In some embodiments, the delivery device is a cannula. In some embodiments, the cannula is a needle. In some embodiments, the delivery device further comprises a thermal jacket.

In some embodiments, the system further comprises a container configured for insertion in the repository. In some embodiments, the container is disposable. In some embodiments, the container comprises pre-mixed solution. In some embodiments, the container comprises a container identifier. In some embodiments, the container identifier is selected from the group consisting of a radio-frequency identification (RFID) tag, a chip, or a barcode.

Certain embodiments of the invention are directed to methods for generating a slurry. The methods comprise receiving a solution in a slurry generator, and generating slurry from the solution, wherein the slurry comprises ice particles capable of flowing through a cannula.

Methods of the invention further comprise preparing the solution. The solution may comprise liquid water and one or more additives. The one or more additives may affect tonicity and/or flowability of the slurry. In some embodiments, the one or more additives comprise one or more of a salt, a sugar and a thickener. In some embodiments, Additives can include any substance on the FDA GRAS list for example, sodium chloride, glycerol, sodium carboxymethylcellulose (CMC), dextrose, xanthan gum, glycerin, polyethylene glycol, cellulose, polyvinyl alcohol, polyvinylpyrrolidone, guar gum, locust bean gum, carrageenan, alginic acid, gelatin, acacia, and carbopol.

In some embodiments, receiving the solution in the slurry generator comprises inserting a container in a repository of the slurry generator. In some embodiments, the container is disposable. In some embodiments, the container comprises pre-mixed solution. In some embodiments, the solution can be created and customized on demand. In some embodiments, the container comprises a container identifier selected from the group consisting of a radio-frequency identification (RFID) tag, a chip, or a barcode.

Methods of the invention further comprise generating the slurry in an aseptic systems and methods. For example, sterility may be maintained by using sterilized materials and using aseptic transfer methods.

In some embodiments, generating the slurry comprises cooling solution in the slurry generator. In some embodiments, the slurry is generated in the slurry generator when a nucleation event generates ice particles. In some embodiments, the ice nucleation occurs at about 0° C. to about −15° C. In some embodiments, the method further comprises switching the system to a maintenance mode when a temperature of the slurry solution reaches a certain temperature, for example at a temperature at or below 0° C. In some embodiments, maintaining the temperature of the solution provides a slow, controlled formation of ice particles. In some embodiments, inducing ice nucleation further comprises inducing ice nucleation in zones around particulates. In some embodiments, the method further comprises preventing accumulation of particulates and unwanted formation of crystals by generating the slurry in a system with smooth surfaces. In some embodiments, the particulates may have a mechanical function such as preventing clumping in the system by agitating the solution. In some embodiments, the slurry is generated in a continuous flow system. In some embodiments, the slurry is generated in an agitated system. In some embodiments, the slurry is generated in a hybrid system having continuous flow and agitation.

Methods of the invention further comprise aseptically transferring slurry from the slurry generator. In some embodiments, the aseptic transfer comprises automated aseptic transfer from the slurry generator to a sterile delivery device using a luer connection. In some embodiments, the delivery device is disposable. In some embodiments, the delivery device is a handheld device.

In some embodiments, methods of the invention further comprise injecting slurry into a subject. In some embodiments, the slurry is injected into subcutaneous fat of the subject. In some embodiments, the cannula is a needle. In some embodiments, the needle has a gauge size of about 8 G to about 25 G.

The present invention additionally provides methods and systems for controlling generation of ice particles to produce a slurry for injection by varying inputs, e.g., solution and solution ingredients, and process parameters. Additionally, by controlling nucleation, the initial process for forming ice particles, the ice particles are formed in a slow, controlled manner. This controlled formation allows growth of globular, or spherical, ice particles to a desired size and even dispersion of the ice particles throughout the slurry. Even dispersion and uniformity in shape and size of the ice particles prevent blockages in the generation system as well as the selected delivery device used for delivery of the slurry to a subject, for example, a cannula.

The slurry may be used for a wide range of health-related applications. For example, the slurry may be injected into subcutaneous fat or adipose tissue. Once injected, the slurry causes cryolipolysis, or cell death by freezing of fat cells. As such, targeted removal of subcutaneous fat is possible using the injected slurry. In addition, the slurry may be injected into certain tissues or organs to directly reduce inflammation. The slurry may be administered by any suitable method, such as injection into a subject's body by a cannula such as a needle. Injection of the slurry may be targeted to certain areas of the body by a trained professional in a single session, or multiple sessions. Especially in the context of reducing adipose tissue such as for improving cosmetic appearances or reducing obesity, extensive surgery, long treatment times, and consulting with a plastic surgeon may be avoided.

### BRIEF DESCRIPTION OF THE DRAWINGS

FIG. 1 is a flowchart of a method according to an embodiment of the invention.

FIG. 2 depicts a system in accordance with certain embodiments.

FIG. 3 depicts an exemplary embodiment of a handheld slurry generation system.

FIG. 4A and FIG. 4B depict the disposable architecture for the cartridge of a handheld slurry generation system.

FIG. 5 depicts an exemplary delivery device for a handheld slurry generation system.

FIG. 6-G depicts an exemplary process for utilizing a handheld slurry generation system.

FIG. 7 shows an embodiment of a system for generating a slurry.

FIG. 8 shows a perspective view of a system for generating a slurry.

FIG. 9 shows an embodiment of a system for generating a slurry.

FIG. 10 shows a reservoir for a slurry.

FIG. 11 shows an internal cross section view of a reservoir for a slurry

FIG. 12 shows a lid for a reservoir for a slurry

FIG. 13 shows a cartridge for a slurry.

FIG. 14 shows a collapsible member.

FIG. 15 shows a handheld device for administering a slurry.

FIG. 16 shows a handheld device for administering a slurry.

FIG. 17 shows a syringe for administering a slurry.

FIG. 18 diagrams a slurry generation system of the present invention.

FIG. 19 diagrams a slurry generation system of the present invention and authentication of a container to be used with a slurry generation device.

FIG. 20 shows an exemplary embodiment of a slurry generation system.

FIG. 21 shows an exemplary embodiment of a close-up view of a container inserted in a slurry generation device.

FIG. 22 shows an enlarged view of a connection between a container and a slurry generation device and initial RFID reading to determine authenticity of the container.

FIG. 23 shows an enlarged view of a connection between a container and a slurry generation device and initial barcode reading to determine authenticity of the container.

FIG. 24 shows an enlarged view of a connection between a container and a slurry generation device and initial chip reading to determine authenticity of the container.

FIG. 25 shows continuous cooling of slurry over time and formation of ice crystals from slurry solution with fewer free-floating particles.

FIG. 26 shows continuous cooling of slurry over time and formation of ice crystals from slurry solution with a moderate amount of free-floating particles.

FIG. 27 shows continuous cooling of slurry over time and formation of ice crystals from slurry solution with a greater amount of free-floating particles.

FIG. 28 depicts a system of the invention in accordance with certain embodiments.

FIG. 29 shows an embodiment of an ice needle slurry generation system.

FIG. 30 shows an exterior view of an embodiment of an agitated cartridge.

FIG. 31 shows an internal assembly view of an embodiment of an agitated cartridge.

FIG. 32 shows an agitator assembly view.

FIG. 33 shows a plunger assembly view.

FIG. 34 shows an end cap assembly view.

FIG. 35 shows a cartridge cover assembly view.

FIG. 36 shows an internal view of an agitated cartridge

FIG. 37 shows a cutaway internal view of the agitated cartridge.

### DETAILED DESCRIPTION

Methods and systems for generating slurry are provided. In one application, the slurry may be injected into subcutaneous fat of a subject to facilitate weight reduction or improve cosmetic appearances via cryolipolisis. Systems for generating a slurry comprise a repository for receiving a solution, a generator for generating the slurry from the solution, and a port for transferring the slurry from the system. Methods for generating a slurry comprise receiving a solution in a slurry generator, and generating slurry from the solution, wherein the slurry comprises ice particles capable of flowing through a cannula. Systems include continuous flow systems, agitation systems, and hybrid continuous flow and agitation systems.

Types of systems for generating a slurry may include an agitation system, such as an agitated syringe; a continuous flow system; and a hybrid continuous flow and agitation system. Other types of systems for generating a slurry include a blender and/or grinder, a scraped surface system similar to or the same as an ice cream maker or slushy maker. Also disclosed is a system for generating ice in a substantially solid state, for example an ice needle.

Slurry generators may include a cold extraction system, a mixing system and a storage system. The various system can be characterized by the location in which these actions occur. For example, in the agitated syringe system and the ice needle system, each of the cold extraction system, mixing system, and storage system are in the same location. In a continuous flow system, a hybrid system, and a scrape surface system, the cold extraction system is located in a first location, and mixing and storage systems may be located in a second location. In a blender and/or grinder system each of the cold extraction, mixing, and storage systems are in separate locations. An agitation system and a hybrid system further include an agitator, for example, an impeller which can be located in any of the cold extraction, mixing or storage system locations.

Slurry generators can also be characterized by where a nucleation event occurs. Nucleation is the initial process at which ice crystals begin to form, and can be either on a surface, for example a surface of a system component, or in solution. In an agitation system, a continuous flow system and a hybrid system, nucleation occurs in solution. In a scraped surface system and an ice needle system, nucleation occurs at a surface of the system, for example at the surface of a container or a tube. Nucleation can be initiated, for example, via a nucleator such as a pinch valve, or nucleation can be spontaneous.

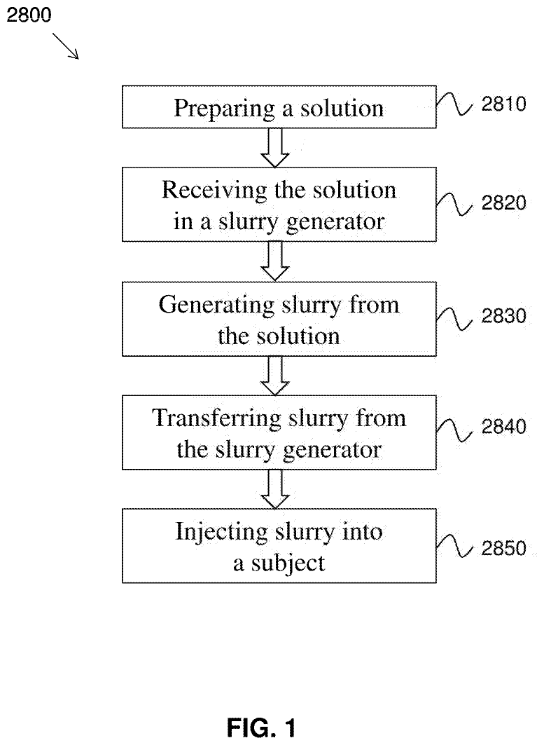

Methods of the invention are directed to generation of a slurry. FIG. 1 is a flowchart of an exemplary embodiment of a method of the invention. The method comprises 2810 preparing a solution. Components of the solution, for example, liquid water and one or more additives, are selected to generate slurry with desired properties. The method further comprises 2820 receiving the solution in a slurry generator. The solution may be inserted into the slurry generator in any suitable manner. For example, the slurry may be in a container and the container may be inserted into a repository of the slurry generator. Alternatively, the solution can be provided directly to the slurry generator via a port. The method further comprises 2830 generating slurry from the solution. Parameters of the slurry generator are adjusted to generate the slurry from the solution. For example, the temperature and flow rate are controlled to cool the solution and generate the slurry. The method further comprises 2840 transferring the slurry from the slurry generator. Once generated, the slurry is ready for injection into a subject. The slurry is transferred from the slurry generator to a delivery device, for example via a port for aseptic transfer. The delivery device may be a cannula, such as a needle. The method further comprises 2850 injecting the slurry into a subject. The slurry may be injected by a healthcare professional in a manner consistent with a treatment plan, such as for injection into subcutaneous fat for fat removal.

### Systems for Slurry Generation

Embodiments of the invention are directed to systems for generating slurry. Systems for generating a slurry include an agitation system such as an agitated syringe, a continuous flow system, and a hybrid system. Other types of systems for generating a slurry include a blender and/or grinder and a scraped surface system similar to or the same as an ice cream maker or slushy maker. Also disclosed is a system for generating ice in a substantially solid state, for example an ice needle. The systems comprise various attributes including but not limited to ease of dispensing of slurry, sterile and disposable components of the system having fluid contact, time between required maintenance, size, set up time and ease, efficient use of solution, open/closed system, cool-down times, materials used, locations of cold extraction, mixing and maintenance of slurry, volumes of slurry generated, and level of control over various properties of the slurry.

Systems for generating slurries additionally provide parameters and ranges that can be controlled and optimized. For example, in a hybrid system each of the length of uncooled tubing, nucleation temperature, slurry flow rate, slurry dispensation rate, tubing geometry including tubing length, diameter, agitator speed, agitator geometry such as paddle geometry, properties of surface materials, gas flow rate, temperature sensor positioning, slurry tank straw positioning, and slurry profiles including cooling temperature, maintenance temperature, and growth temperature can be varied and optimized.

In an agitated syringe system, each of the total volume of slurry, aspect ratio of the system, agitator shape including agitator pitch, angle, and width, agitator cone shape, usable volume of slurry, coolant temperature profile, agitator speed profile including cooling, growth, and maintenance, volume to need transition geometry, syringe angle, and maintenance period can be varied and optimized.

By optimizing process parameters, the followed parameters can also be controlled and optimized including amount of usable slurry, stratification of crystals, degree of ice coagulation, ice growth rate, ice coefficient, crystal size, crystal shape and smoothing, ingress of particulates, amount of air entrained, maintenance period, and particulate generation.

### Continuous Flow System

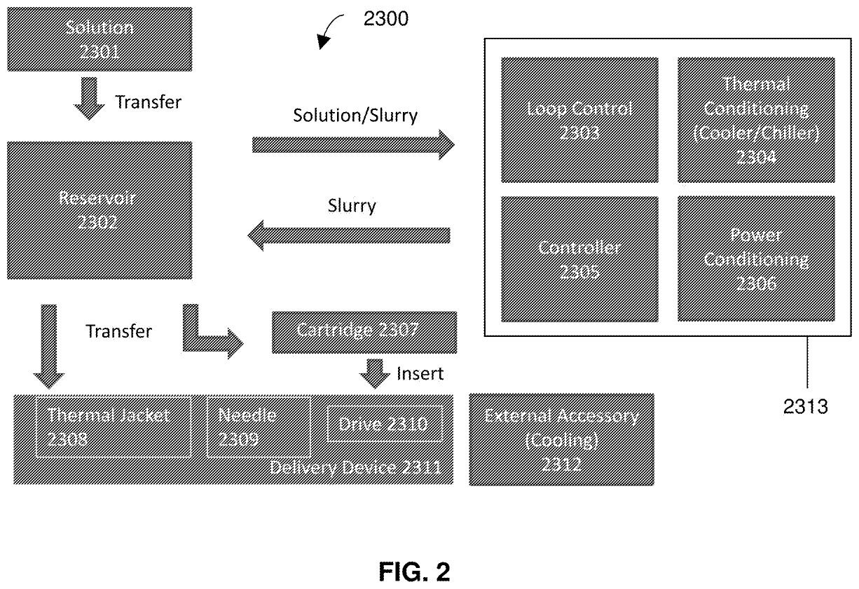

FIG. 2 shows an exemplary continuous flow system 2300 for generating a slurry. In the system 2300 a solution 2301 is transferred to a first reservoir 2302. The solution is processed by a slurry generator 2313 comprising one or more of a loop control 2303, thermal conditioning 2304, a controller 2305, and a power conditioning 2306, to generate a slurry. The reservoir 2302 may be located within the slurry generator or may be external to the slurry generator. The generated slurry may also flow through the generator 2313 in order to maintain continuous flow of the slurry. In some embodiments, the flow rate can be about 20 ml/min to about 200 ml/min.

The slurry in the reservoir 2302 is then transferred to a delivery device 2311 comprising one or more of a thermal jacket 2308, a cannula such as needle 2309, and a drive 2310. Optionally, the slurry in the reservoir 2302 may be transferred to a cartridge 2307 which enters the delivery device 2311. The system may comprise an external accessary 2312, for example, a refrigerator, to maintain the temperature of various components of the system such as cartridge 2307 or the delivery device 2311 before and/or after it has been loaded with slurry.

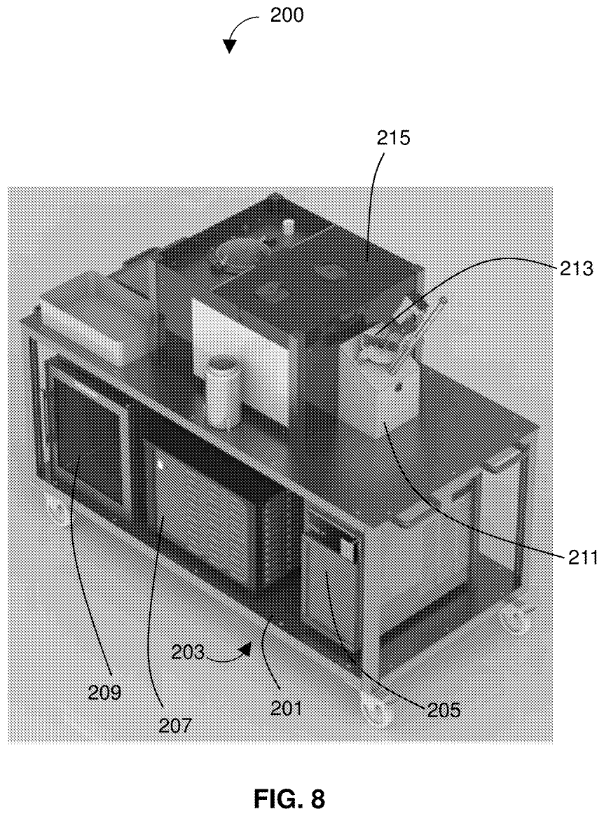

FIG. 8 shows a perspective view of an exemplary continuous flow system 200 for generating a slurry. In this embodiment, system 200 is provided with a base station 201 with reservoir 211. In various embodiments, base station 201 may include wheels such that base station 201 and other components of system 200 may be easily transported. In this example, system 200 further includes a cooling device 203 for cooling a solution used for generating a slurry. As shown, cooling device 203 includes a chiller 205 and a cooler 207. Base station 201 is also provided with a refrigerator 221, which may be used to maintain a temperature of various components such as syringes, insulating thermal jackets for syringes, or cartridges.

System 200 further includes circulating system 215, which includes a pump in fluid communication with the reservoir 211 via tubing for circulating the solution at least from the reservoir 211 to the cooling device 203. System 200 may also include a nucleator (not shown) connected to the circulating system 215 for nucleating the solution such that ice particle formation is initiated. In some embodiments, the nucleator may be provided in a cartridge in fluid communication with circulating system 215, such that a continuous flow of slurry may pass through the cartridge to and from the circulation system 215. In some embodiments, nucleation occurs spontaneously.

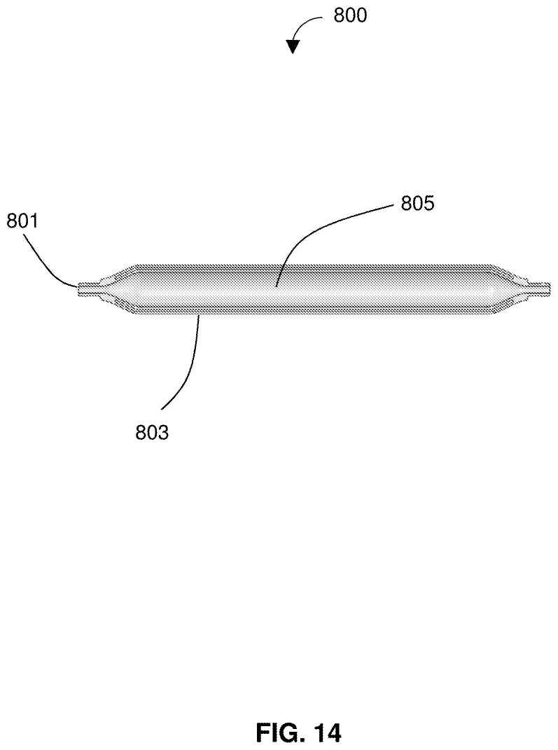

FIG. 14 shows an example of a collapsible member 800, which may be provided in tubing within a cartridge and/or tubing within the system for a slurry utilizing continuous flow. In this illustration, collapsible member 800 includes an elongated body 803. In certain embodiments, collapsible member 800 may be in fluid communication with the circulating system of any continuous flow or hybrid systems described herein, and a force such as a vacuum force, pinching force, or any suitable force may be applied to collapsible member 800 to induce nucleation. As such, collapsible member 800 may be advantageously provided to the system for generating a slurry to induce nucleation and provide a consistent slurry suitable for administration to a human subject.

Elongated body 803 may be of any suitable shape, such as a bulb shape, an elongated bulb shape, a tubular shape, or the like. As shown, collapsible member 800 includes an opening 801 with a first diameter that is less than a second diameter 805 of the elongated body 803. Opening 801 may be in fluid communication with a circulating system of the system for generating a slurry. In various embodiments, collapsible member 800 may be included in a cartridge for containing and/or administering the slurry. In other embodiments, collapsible member 800 may be provided in a handheld device to which a cartridge may be connected to in order to administer the slurry, or provided in various placements in fluid communication with the circulating system.

By providing a first diameter at opening 801 that is smaller than second diameter 805 of elongated body 803, collapsible member 800 may allow a maximal velocity in the widened region at second diameter 805. Such increased velocity may reduce agglomeration and stratification of the slurry by reducing melting or heating of ice particles, and facilitate a continuous flow of slurry.

The collapsible member may be of any suitable size, length or dimension. For example, a collapsible member shaped as an elongated bulb may be between 2 and 12 inches long, have a first diameter 801 of 0.1 to 0.5 inches and a second diameter of 0.2 to 3 inches.

Reservoir 211 can be shaped and positioned such that gravity allows for the solution/slurry to be maintained at a level vented to air. This configuration additionally provides for minimizing slurry waste.

Systems for generating the slurry may comprise handheld systems. Handheld systems may provide a base station providing drive to the delivery device rather than a motor within the delivery device. Handheld systems can be rechargeable. Handheld systems can be of suitable size and weight to be usable by a clinician.

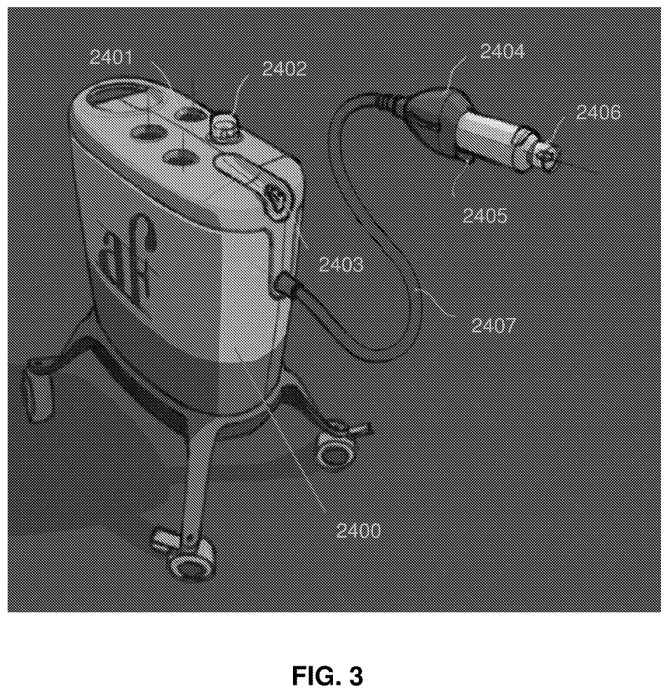

FIG. 3 shows an exemplary handheld continuous flow system 2400. In the handheld system 2400 a cooling bay 2401 for individual cartridges 2402 is provided with a cooling dock 2403 for the delivery device. The delivery device comprises a gear box 2404 to drive the slurry from the inserted cartridge 2406. Power is delivered to the gear box through a double wire drive 2407 connected to the base of the handheld system 2400. The delivery device further comprises a rechargeable cooling jacket or cooling connection 2405 to maintain the temperature of the inserted cartridge 2406.

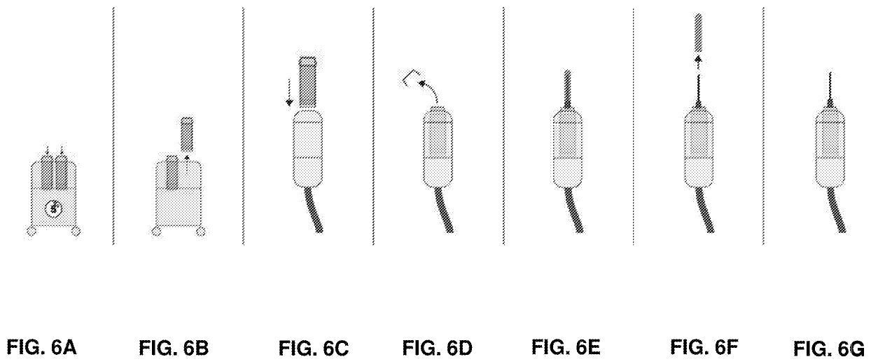

FIG. 6A-G shows an exemplary process for utilizing a handheld device. As shown in FIG. 6A, two sealed cartridges are placed in a base station. In FIG. 6B, the cartridge is removed from the base station and loaded into a handheld delivery device, as shown in FIG. 6C. As shown in FIG. 6D the seal is removed from the cartridge, and in FIG. 6E a needle with a needle cover is attached to the handheld delivery device. In FIG. 6F, the needle cover is removed from the needle and in FIG. 6G, the system is prepared for administering the slurry.

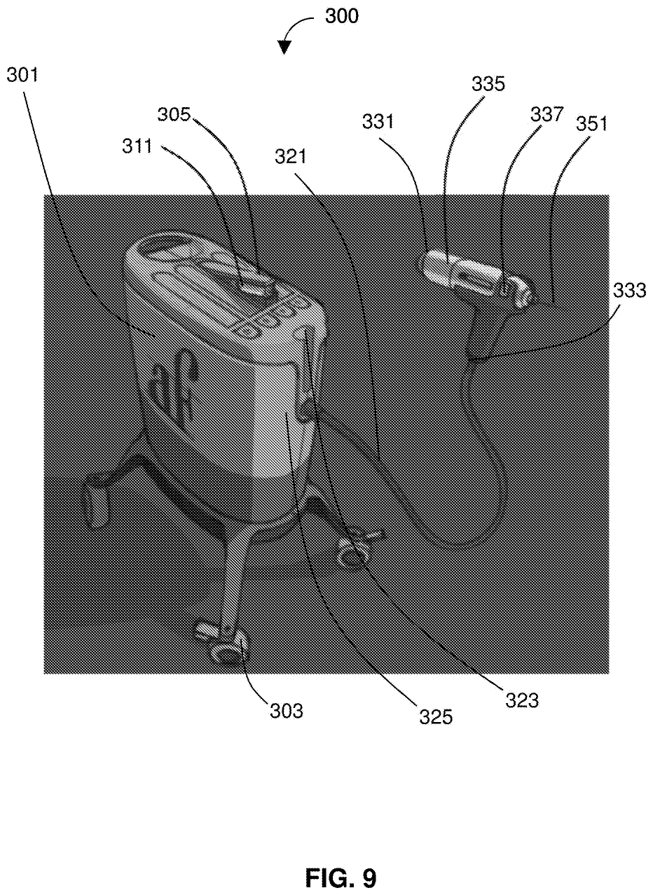

FIG. 9 shows another embodiment of a handheld continuous flow system 300 for generating a slurry 305, which includes a handheld device 331 for administering slurry 305 to a subject. In this embodiment, the base station 301 with wheels 303 may house part of all of the components of system 300 such that system 300 may be easily transported to optimize the experience of a subject treated with slurry. In this example, system 300 may generate a slurry at continuous flow, such that a continuous flow is maintained throughout a circulation system of system 300, as well as at one or more ports 305 and one or more cartridges 311, which are each connected at a port 305. Base station 301 may include multiple ports 305, each of which may be connected to one or more cartridges 311. In this example, system 300 may generate a volume of slurry that is circulated through each of the cartridges 311 such that a slurry suitable for administration to a human subject is maintained and a particular volume of slurry may be received at each cartridge via port 305. Cartridge 311 may then be detached from port 305 and attached to handheld device 331 to be administered to the subject through needle 351. Any suitable number of ports and cartridges may be used.

System 300 provides a single readily moveable unit for treatment of a subject, with each cartridge 311 accessible to be dispensed with no or minimal downtime. As such, the treatment time and amount of discomfort of a subject may be reduced and the experience of the treated subject is improved. For example, a slurry may be generated and maintained at continuous flow throughout system 300, including cartridges 311. Cartridges 311 may each be set to allow a volume of slurry to be available when a cartridge 311 is detached from port 305. As an example, each cartridge 311 can provide 30 mL of slurry. For a treatment requiring four separate injections in four separate locations, cartridges 311 may each be maintained at parameters suitable for injection throughout the time of treatment. Each cartridge may be detached from a port 305, attached to handheld device 331, and the slurry administered to the subject in rapid succession. In addition to minimizing treatment time and downtime in the procedure, system 300 helps provide a consistent slurry by reducing melting of ice particles in the slurry. For example, if multiple syringes of slurry were simultaneously prepared and injected into a subject in sequence, the consistency within the first and last syringes may differ greatly. In contrast, successive administration of slurry may be performed with minimal changes in parameters due to the continuous flow of system 300.

As shown, base station 301 includes a drive motor 325 that is connected to handheld device 331 via wire drive 321. Handheld device 331 includes body 335, which is connected to the wire drive 321 at a connection 333. Handheld device 331 also includes one or more actuation buttons 337, which may be used to control an actuation valve within body 335, for preventing, allowing, or controlling a flow of slurry to needle 351. For example, wire drive 321 may be controlled at base station 301 by drive motor 325 to apply a pressure to the slurry within a cartridge 311 attached to handheld device 331. For injection to a subject, actuation buttons 337 may be used to release the slurry at a specific flow rate. By providing a slurry at consistent parameters and finely controlling the flow rate of slurry to a subject, system 300 minimizes irritation and pain, enhances tissue permeability, and reduces possible tissue damage (e.g., inflammation effects, including heat, redness, swelling, and pain).

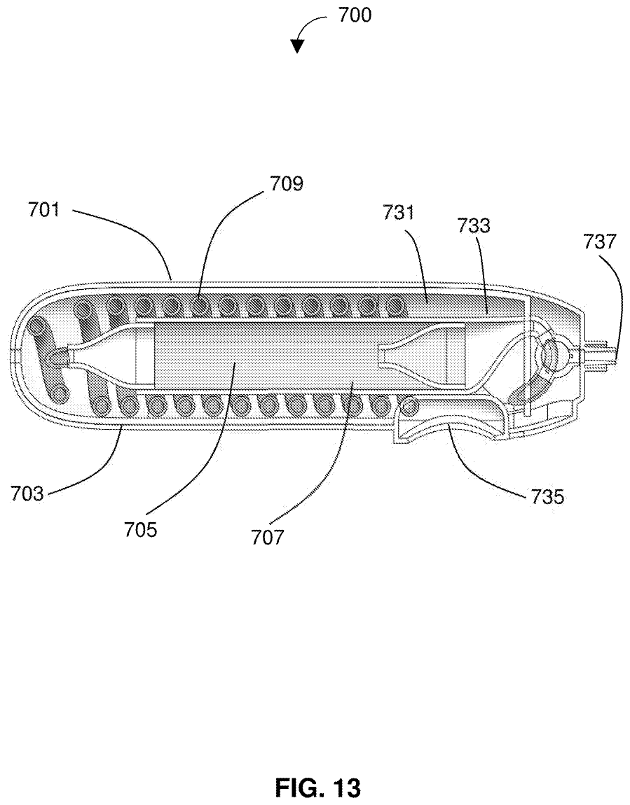

FIG. 13 shows a cartridge 700 for a slurry 705. A continuous flow of slurry 705 may be provided throughout cartridge 700 when the cartridge 700 is connected to a system for generating slurry such as continuous flow system of FIG. 9.

As shown, cartridge 700 includes insulation 703, handheld reservoir 707, which contains slurry 705, tubing 709, needle housing 737, and connection opening 735. In one example, slurry 705 may be received by cartridge 700 at connection opening 735 when the connection opening 735 is placed in fluid connection with a port that is in fluid communication with the circulating system. For injecting the slurry 705 to a subject, a needle of suitable gauge size may be connected to needle housing 737.

In this example, tubing 709 is sterile tubing in fluid communication with sterile handheld reservoir 707. Sterile tubing 709 is in contact with coolant reservoir 731, which contains a coolant 733. In various embodiments, coolant 733 may be in fluid connection with a coolant contained in the cooling device. For example, a volume of coolant may be provided to the cooling device of the base station and at least a portion of such coolant circulated to cartridge 700. By contact of tubing 709 with coolant 733, heat may dissipate into coolant 733 and facilitate maintaining the slurry 705 at a consistent temperature and at consistent parameters suitable for human administration. The slurry 705 does not itself contact coolant 733. Only tubing 709 contacts coolant 733 to allow heat dissipation. In addition, by providing cartridge 700 with a length of wound tubing 709 in contact with coolant 733, a solution for generating the slurry 705 may be circulated via peristaltic pumping in the continuous loops of wound tubing, which may induce nucleation. The tubing may be any suitable tubing of a suitable length, for example, silicone tubing. Cartridge 700 may comprise a plunger configured to dispense slurry through reservoir 707 similar to a syringe.

Cartridge 700 comprising continuous flow allows for a consistent slurry 705 to be maintained at each cartridge 700 and such slurry 705 may be administered to a subject substantially at the same parameters maintained within the circulating system. Consistent delivery of slurry 705 to a subject may maximize the effectiveness of a treatment and minimize variables when planning subsequent treatments based on the results attained.

### Agitation System

One embodiment of a slurry generation system is an agitation system, such as an agitated cartridge or agitated syringe.

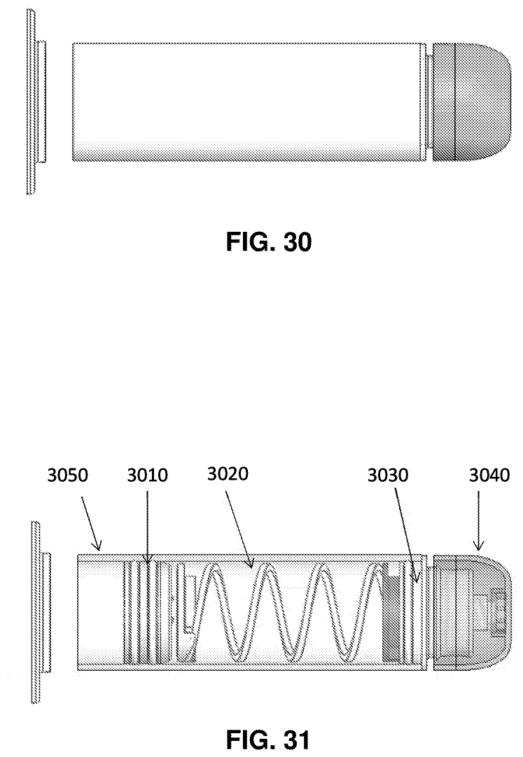

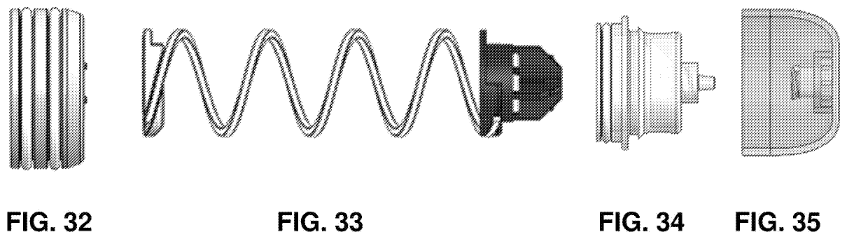

FIG. 30 shows an exterior view of an embodiment of an agitated cartridge. FIG. 31 shows an internal assembly view of the agitated cartridge. The internal assembly view includes a plunger assembly 3010 (FIG. 32), agitator assembly 3020 (FIG. 33), end cap assembly 3030 (FIG. 34), and cartridge cover 3040 (FIG. 35) inside the cartridge housing 3050.

In some embodiments, the plunger assembly comprises a standard O-ring design. In some embodiments, the plunger assembly comprises protrusions on the face of plunger providing a bearing surface for agitator rotation, and a hollow interior providing minimal gap between magnets for maximized coupling torque.

The agitator assembly, such as an agitator coil, optimizes ice slurry homogeneity during operation. Compressibility of the coil allows for the least dead space volume inside of the cartridge. In some embodiments, the agitator assembly may be constructed of any suitable material, for example, a PETG material.

In some embodiments, the end cap assembly has a standard O-ring design with a luer lock design that minimizes dead space volume inside of off-the-shelf needle hubs. In some embodiments, the end cap may be constructed of an optically clear Topaz material to allow for visual bubble detection inside of the slurry. In some embodiments, the cartridge cover assembly provides a sterile barrier compatible with an off-the-shelf luer tip cap.

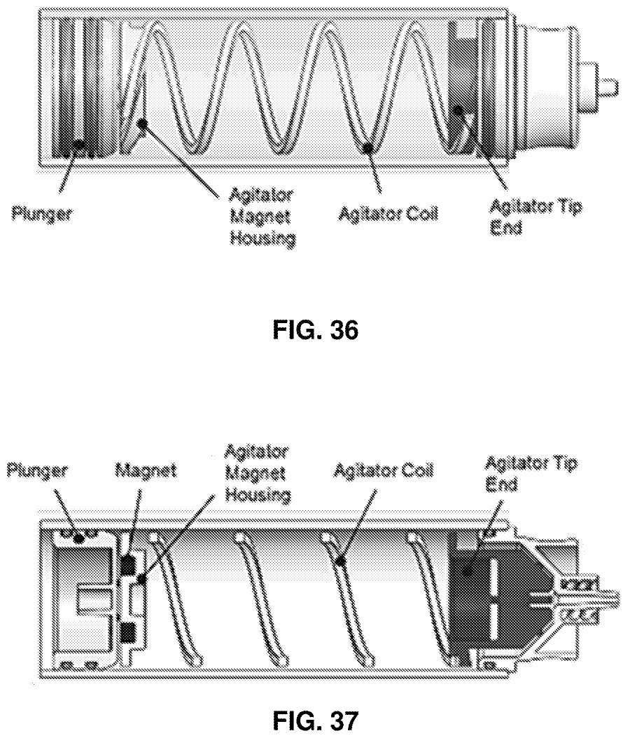

FIG. 36 shows an internal view of the assembly of an agitated cartridge, and FIG. 37 shows the corresponding cutaway view of the internal assembly of the agitated cartridge. The interaction between the plunger, agitator magnet housing, magnet, agitator coil, and agitator tip end or end cap is shown.

The agitated cartridge design relies on an internal agitator to keep the slurry well mixed, preventing agglomeration and stratification. Torque is transferred to the agitator for rotation using a magnetic coupling that acts axially through the front face of the plunger. This allows for torque transfer without incorporating dynamic shaft seals into the sterile boundary. In some embodiments, one half of the magnetic coupling is embedded in the rear of the agitator, and the other half of the coupling remains part of the handheld, which provides the rotational torque.

The magnets may provide sufficient torque to agitate the slurry while keeping the axial attraction force at a minimum to limit friction losses and minimize risk of generating internal particulates at the bearing surfaces. In some embodiments, the stack-up of material thicknesses in the plunger and agitator magnet housing produces a minimum gap distance of approximately 2-3 mm between the magnets.

The syringe may be graduated, agitated, insulated, and filled repeatedly. Ice coefficient may be kept consistent to allow for consistent delivery of slurry throughout the predicted workflow. The agitated syringe system may use a removable cooling jacket which can be stored separately to the syringe. By using multiple jackets, multiple slurry samples may be taken with no time in between to re-cool the syringe. The agitated syringe system may have visible markings for volume dispensed. The agitated syringe system may be made of components that are cleanable or replaceable. The agitated syringe system allows filling with slurry without compromising the ice coefficient of slurry within the slurry generation system. The agitated syringe system shall be possible to operate using a standard syringe pump. Using a syringe pump allows for accurate control of the dispense rate.

The agitator may have any suitable geometry. In some embodiments, the agitator geometry has a ribbon profile. In some embodiments, the ribbon profile is selected from rectangular, square, and round profiles from previous circulation tests. In some embodiments, the ribbon mixer extended into the syringe cone with simple crosshair features that attempted to leave large windows of open area for slurry to circulate through. In some embodiments, the agitator geometry has a closed window design.

In some embodiments, the agitation speed is about 500 RPM to about 2500 RPM. In some embodiments, the agitator speed is about 2000 RPM. Agitator speed has a relationship with the maintenance temperature. In some embodiments, the maintenance temperature setpoint is about −3 C to about −5 C. Testing shows that colder temperatures or lower speeds cause a steeper increase in torque, and a higher torque value for any given ice coefficient. For example, with a −5° C. setpoint and 750 RPM, a 30% slurry had a torque close to 50 N-mm. A similar 30% slurry generated with −5° C. setpoint but 2000 RPM has a torque approximately 21-22 N-mm. Testing shows that unbalanced combinations create a thermal gradient with a colder region along the walls of the syringe that the agitator cannot mix evenly without moving faster. Crystals forming along the cartridge wall increase the torque on the agitator until some balance is reached. Testing shows that a 30% IC slurry correlates to a measured torque around 22 N-mm when agitation speed is sufficiently high (depending on the maintenance temperature).

In addition to steady agitator speeds, pulsing agitation may be used in the invention. In some embodiments, pulsing agitation (varying agitation speeds and durations) is 500 RPM for 9 s followed by 2500 RPM for 1 s; 500 RPM for 5 s followed by 2500 RPM for 5 s; or 1000 RPM for 5 s followed by 2500 RPM for 5 s. In some embodiments, a maintenance temperature of about −5° C. to about −3° can be set in combination with an about 2000 RPM agitation speed. The maintenance setting can be varied depending on the amount of time between slurry generation completion and the treatment. Any type of agitation and/or agitation settings and combinations thereof may be employed.

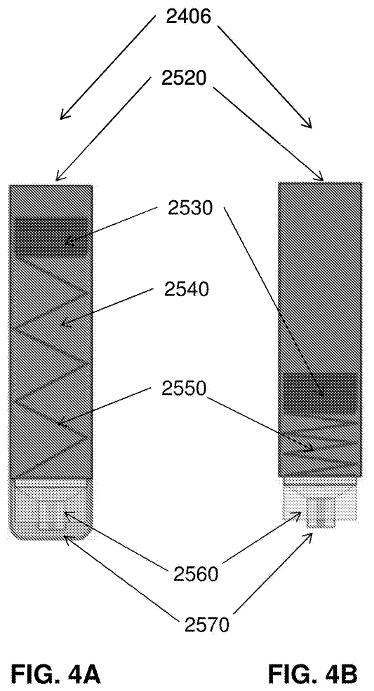

FIG. 4A and FIG. 4B depict an embodiment of a cartridge 2406 for a handheld system. The cartridge 2406 comprises a container 2520 holding an initial volume of slurry 2540. When a plunger 2530 is depressed, an agitator 2550 within the cartridge collapses, similar to a compression spring, agitating the slurry prior to expression the slurry. Thereafter, the cartridge can be removed from the handled system and disposed. The cartridge further comprises a finger flange 2560 and luer 2570 to enable flow of the slurry in and out of the cartridge.

FIG. 4A and FIG. 4B depict an embodiment of a cartridge 2406 for a handheld system. The cartridge 2406 comprises a container 2520 holding an initial volume of slurry 2540. When a plunger 2530 is depressed, an agitator 2550 within the cartridge collapses, agitating the slurry prior to expression the slurry. Thereafter, the cartridge can be removed from the handled system and disposed. The cartridge further comprises a finger flange 2560 and luer 2570 to enable flow of the slurry in and out of the cartridge.

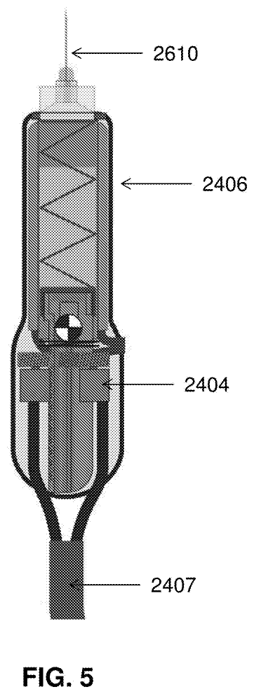

The cartridge and handheld of FIGS. 4A, 4B and 5 can be used with a continuous flow or hybrid system to agitate the slurry prior to and during delivery to a subject.

FIG. 5 shows an exemplary delivery device for a handheld system. The delivery device comprises the cartridge 2406 inserted into a syringe system 2610. The delivery device further comprises gear boxes 2404 deriving power from a double wire drive system 2407. Once power is delivered to the gear boxes 2404, for example via engagement of an activation button, an amount of slurry is released from the syringe system 2610 and/or power is delivered to the agitator.

### Hybrid System

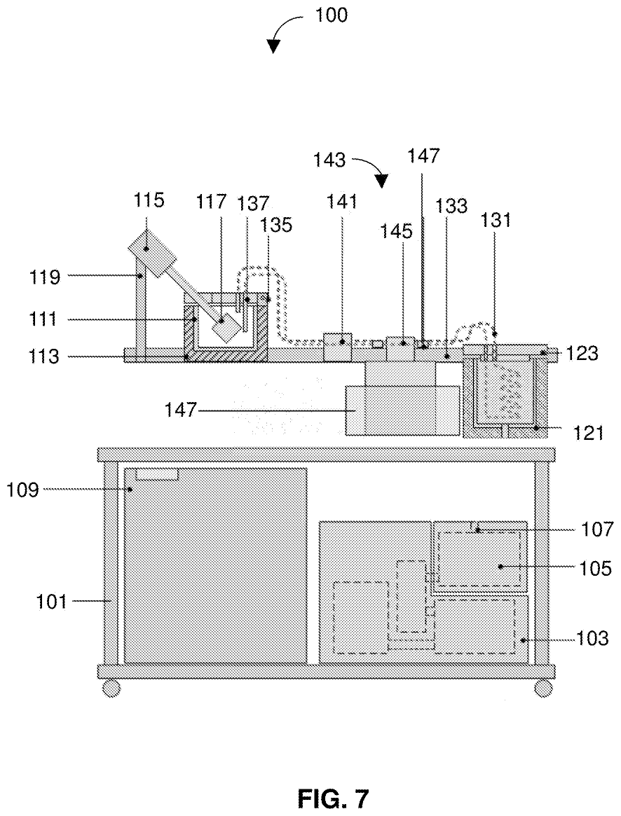

FIG. 7 shows an exemplary embodiment of a hybrid system 100 for generating a slurry. System 100 includes a base station 101 with a slurry reservoir 111 and a cooling device 103. Base station 101 may optionally include refrigerator 109, which may be used to contain pre-prepared solution, constituents of a solution, syringes for injection, thermal jackets for the syringes, and other components that may be used with system 100. When preparing a slurry, a solution used to generate the slurry may be introduced to slurry reservoir 111 and cooled by cooling device 103. As shown, cooling device 103 includes coolant reservoir 105, coolant opening 107, coolant insulation 121, and coolant cover 123. Although the connection is not shown, coolant reservoir 105 is in fluid connection with the portion of coolant reservoir 105 covered by coolant cover 123 and insulated by coolant insulation 121.

System 100 further includes circulation system 143, which includes a pump 145 in fluid communication with the slurry reservoir 111 via tubing 131 for circulating the solution at least from the slurry reservoir 111 to the cooling device 103. The pump 145 may be a peristaltic pump or any other suitable pump that moves the solution or slurry to and from coolant reservoir 105. Tubing 131 may be insulated by tubing insulation 133 to decrease the introduction of heat into the slurry while circulated by circulating system 143.

In this embodiment, insulation 113 is at least partially in contact with slurry reservoir 111. As shown, slurry reservoir 111 is covered by lid 135 with tubing connections 137 to connect slurry reservoir 111 to tubing 131 such that the slurry is in fluid communication with circulating system 143. Lid 135 may house agitator paddle 117. Agitator paddle 117 is connected to and driven by agitator motor 115. Agitator motor 115 may be supported by an agitator support 119. Agitator paddle 117 may agitate the solution or slurry while the slurry is being generated, while the slurry is being maintained, and/or after the slurry is prepared. By agitating the slurry, temperature may be more readily maintained throughout the volume, and agglomeration of ice particles may be reduced as well as stratification of the slurry. A more consistent slurry may be provided by agitation of the slurry as compared to a system lacking agitation. In some embodiments, a cartridge can comprise the agitator, for example as shown in FIGS. 4A and 4B.

System 100 optionally includes nucleator 141 and fluidic connectors 147 for connecting tubing 131 to pump 145. Nucleator 141 is connected to circulating system 143 and may induce nucleation in the solution such that ice particle generation is initiated. Upon nucleation, the circulating system may maintain a continuous flow of the slurry at least from the reservoir to the cooling device. This continuous flow throughout the system helps maintain a consistent temperature of the slurry, which improves the ice coefficient of the slurry, ice particle size, flowability, and effectiveness when administered. Accordingly, a more consistent slurry may be maintained throughout system 100, resulting in a substantial volume of slurry being ready for a treatment. For example, in a treatment involving four separate injections in separate abdominal positions, any variation between the first and last injection may be minimized by the continuous flow. In some embodiments, nucleation occurs spontaneously upon the system/solution reaching a particular temperature.

When generating a slurry, a solution used for generating a slurry may be provided to slurry reservoir 111. In alternative embodiments, components of the solution may be provided to or mixed in slurry reservoir 111. Circulating system 143 may then circulate the solution to and from cooling device 103 via tubing 131 and pump 145. As the solution circulates to and from coolant reservoir 105, which contains coolant that is colder than a temperature of the initial solution, heat from the solution may dissipate into the coolant through the tubing 131 and thereby cool the solution.

Once the solution is cooled to a certain temperature, nucleation may be induced by the nucleator 141 to form ice particles and generate the slurry. In some embodiments, nucleation is spontaneous. Nucleation is the first step in the formation of either a new thermodynamic phase or a new structure, such as by self-assembly or self-organization of ice particles in a solution containing water. Without a nucleation event, generation of the slurry may take longer and may result in an inconsistent slurry that lacks an appropriate ice coefficient, ice particle size, flowability, and effectiveness when administered.

However, nucleation may be induced by various physical, chemical, or other suitable methods. In one example, nucleator 141 may be a mechanical means of inducing nucleation. For example, nucleator 141 may be a pinch valve, in which an operator or a motorized unit may pinch at least a portion of tubing 131 to induce nucleation. In certain embodiments, a collapsible member may be used to induce nucleation, in which a force may be applied to the collapsible member to cause at least a part of it to collapse and thereby simulate a pinching motion and induce nucleation. For example, the collapsible member may be tubing or may have an elongated body of any suitable shape, such as a bulb shape, an elongated bulb shape, a tubular shape, or the like. In this example, a collapsible member in fluid communication with tubing 131, through which the solution or slurry is circulated, may pinch tubing 131 to cause nucleation. A force such as a mechanical force from a motor or a vacuum force may be applied to the collapsible member to cause at least a part of it to collapse.

In various embodiments, a cartridge (not shown) may be attached in fluid communication with the circulating system 143 or slurry reservoir 111 and receive a volume of slurry. The cartridge may then be used to administer the slurry to a subject through a cannula. For example, a cartridge may receive a volume of between 10 mL and 100 mL of slurry and be attached to a handheld unit with a needle of gauge size 18 G. The slurry may then be administered to a subject through the needle. Various cartridges may be used with system 100 and the cartridges may be reuseable or disposable after a single use or more than one use. In one embodiment, the cartridge includes nucleator 141 and the continuous flow of slurry throughout circulating system 143 includes circulation through the cartridge and nucleator 141 within the cartridge. In another embodiment, the nucleator 141 is not within a cartridge and the cartridge simply receives a volume of slurry that may be injected. In one example, the cartridge may include an agitator to prevent agglomeration, reduce temperature differences within the volume, and maintain a consistent slurry through injection.

The solution/slurry flow rate through the system is another parameter that may be selected and adjusted. In some embodiments, the flow rate comprises about 20 ml/min to about 200 ml/min. The generator further allows for stability of the properties of the slurry for the duration of treatment. For example, if treatment time is approximately one hour, the slurry should be stable for longer than one hour. The slurry generation time is another device parameter that may be adjusted. The treatment time and time leading up to treatment with the slurry may be variable per patient situation. The slurry generation time may be any suitable slurry generation time. For example, the slurry generation time may be less than about 10 minutes to about 12 hours. In some examples, a patient may make a last-minute appointment or be a walk-in patient. In such cases, a healthcare professional may desire a quick slurry generation time, such as less than about 10 minutes. Other times, a healthcare professional may know that a patient is scheduled for an appointment first thing in the morning. In such cases, the healthcare professional may want to set a longer slurry generation time in order to prepare the slurry overnight so that the slurry is generated and ready to administer for the early morning appointment. Therefore, the healthcare professional may set a slurry generation time of about 12 hours.

In another embodiment, system 100 may further include a handheld device that is in fluid communication with circulating system 143 or reservoir 111. The handheld device may be connected to a needle of suitable gauge size, through which the slurry may be administered to a subject. In this example, system 100 may provide a continuous flow of slurry to the handheld device and a series of injections may be performed with minimal downtime between each injection, reducing treatment time and minimizing patient discomfort. The handheld unit may include a pump, such as a peristaltic pump for administering the volume of slurry at a controlled rate. The handheld unit may further include an actuation valve to prevent, allow, or control a flow rate of the slurry through the needle. Thus a single handheld unit may be adjusted for various treatments that may differ in administration technique or other considerations.

In another embodiment, a cannula such as a sterile syringe may be used to administer the slurry. A volume of slurry may be received by the syringe prior to administration to s subject. A needle of suitable gauge size may be attached to the syringe, in fluid communication with the syringe body containing the slurry. The syringe may include insulation contacting at least a part of the syringe body. Alternatively, insulation may be provided as a removable thermal jacket that is insulated and may be cooled separately in a refrigerator 109 and slid over a part of the syringe body 1101 prior to being filled with the slurry 1105 and/or administration to a subject. The slurry may be injected by depression of a plunger of the syringe.

In various embodiments, system 100 may further include a control unit 147 that may be connected to and configured to control or adjust parameters of various components of system 100. For example, the control unit 147 may control agitator motor 115, pump 145, fridge 109, cooling device 103, and sensors that may be attached to components such as the reservoir 111, circulating system 143, or cooling device 103.

The control unit 147 may be connected to various sensors throughout system 100, such as sensors for determining agitator revolutions per minute (RPM), circulation RPM, transfer volume, and transfer RPM of the slurry to a cartridge, syringe, or other components. Other sensors may be provided to determine slurry reservoir temperature, coolant tank temperature, coolant volume, cooler temperature, chiller temperature, ice coefficient of the slurry, slurry volume, time until the slurry is ready to be administered to a subject, time lapsed since a solution for generating the slurry has been input, time lapsed since the coolant was changed, interval for inducing nucleation, and relation between activity of the nucleator 141 and ice coefficient.

In various embodiments, the control unit 147 may further include a processor, memory, and optionally a display for visually depicting any of the above sensor outputs, among other information. Any suitable processor, memory, and display may be used. For example, an LCD or LED display may be used to display information such as sensor information, and to monitor generation or maintenance of the slurry.

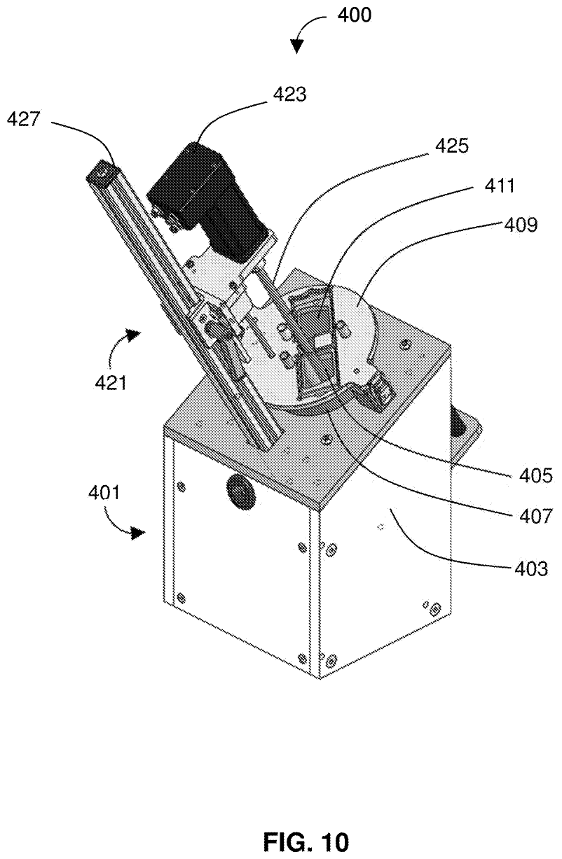

FIG. 10 shows a reservoir 400 for a slurry 405, for example, reservoir 111 of FIG. 7. In this embodiment, reservoir 400 includes housing 401, which contains slurry reservoir 403. Slurry reservoir 403 may contain a solution used to generate a slurry 405 or the slurry 405. Reservoir 400 is in fluid communication via tubing, with at least the circulating system for generating a slurry 405.

In this embodiment, slurry reservoir 403 is covered by lid 409, which is preferably sealed by gasket 407. Lid 409 provides glass cover 411 so that slurry 405 may be viewed without removing the lid 409. Glass cover 411 may also be used to view various sensors that may be housed inside the slurry reservoir 403 and positioned such that a sensor display or output may be viewed through the glass cover 411. As shown, reservoir 400 is connected to agitator 421 via lid 409. Agitator 421 includes agitator motor 423 and agitator drive 425, which is contained inside slurry reservoir 403 in this view, is connected to a paddle for agitating the slurry 405. In this example, agitator 427 is at least partially supported by support arm 427. Agitator 421 may be mounted in various ways and orientations such that agitator 421 may assist in maintaining slurry 405 at parameters suitable for administration to a human subject.

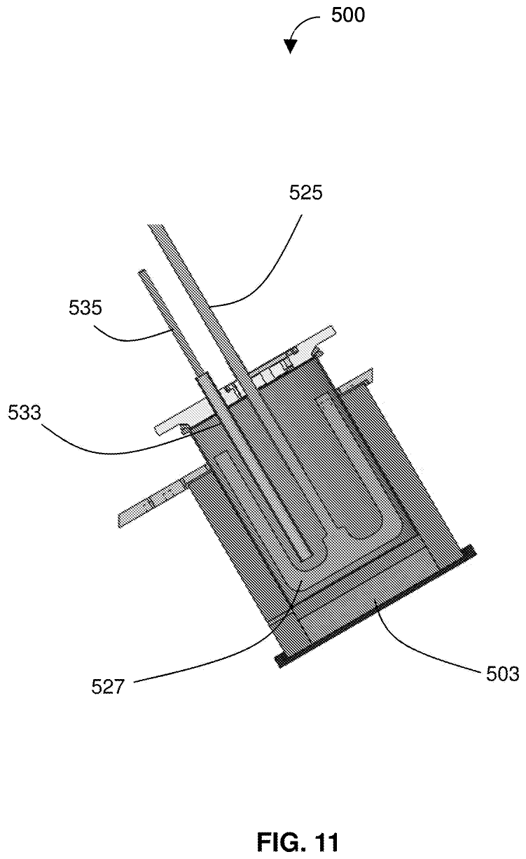

FIG. 11 shows an internal cross section view of a reservoir 500 for containing a slurry or a solution used to generate a slurry, for example reservoir 111 of FIG. 7. As shown, reservoir 500 includes slurry reservoir 503, which is at least partially covered by insulation 529. Slurry reservoir 503 may be at least partially surrounded, completely surrounded, or at least partially covered by insulation 529. In this example, reservoir 500 houses slurry reservoir 503, which is connected to a lid 509 that may be sealed to the slurry reservoir 503 by gasket 507.

Lid 509 may house agitator drive 525, which may be connected on one end to paddle 527 and on another end to an agitator motor (not shown). Lid 509 may further house valves for liquids or gases to be input or removed from the slurry reservoir 503, such as valve 533 to allow slurry to be removed from the reservoir, a valve to allow slurry or a solution to enter the reservoir, and/or a cleaning valve to input a gas or sterilizing solution to clean the slurry reservoir 503 and/or valves, among other components. Lid 509 may further house at least a portion of temperature sensor 535, which may reach into slurry reservoir 503 and detect at least a temperature of the slurry. Cooling of the solution as well as the temperature of the slurry during and after nucleation may be monitored in real-time by temperature sensor 535.

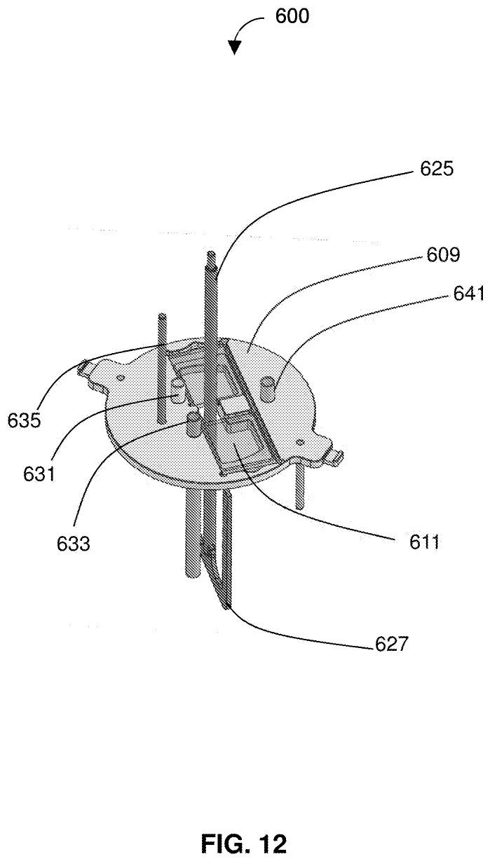

FIG. 12 shows a lid 600 for a reservoir for a slurry. In this embodiment, the lid 600 includes a lid surface 609, which is connected to and houses agitator drive 625 and temperature sensor 635. Agitator drive 625 may be connected on one end to an agitator motor (not shown), and on the other end connected to paddle 627 for agitating a slurry. By agitating a slurry contained within the reservoir, the slurry is able to resist agglomeration. Agglomeration of ice particles in the slurry may cause the slurry to clump and form larger solid or semisolid ice structures, which may inhibit continuous flow throughout the system and/or in a delivery device. Additionally, the agitator also reduces stratification of the slurry and facilitates continuous flow of a more homogenous slurry. Stratification includes the formation of layers of water masses with different properties such as salinity and may cause formation of a layer of ice particles above a layer of water with less or no ice particles, which may not provide a consistent slurry. An inconsistent slurry may hinder the effectiveness of the slurry, such as for removing adipose tissue.

Lid 600 also includes glass cover 611, through which the slurry may be viewed inside the reservoir, and cleaning valve 641. Cleaning valve 641 may be used to input a liquid or gas in to the reservoir. For example, cleaning valve 641 may be used to input a gas or sterilizing solution to clean the reservoir and/or valves 631 and 633, among other components.

### Ice Needles

Also disclosed is a system for generating slurry in a substantially solid state, for example an ice needle. In an ice needle system, solution is frozen fully within a tube and high pressure is used to force the ice out of the tube. With a suitable sizing of the tube, high ice coefficients may be achieved through small ice needles. Further, if ice is fully frozen within the tube, concerns about pauses in the flow blocking the tubing may be prevented.

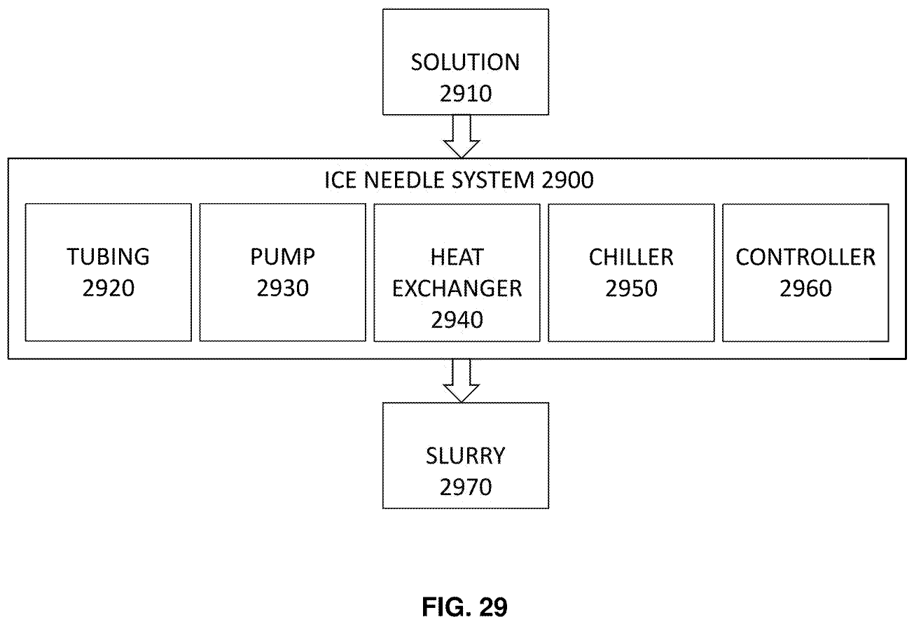

FIG. 29 shows an embodiment of an ice needle system 2900. The inlet and outlet may be any suitable container, such as a solution container. The solution 2910 is pumped through tubing 2920 by a pump 2930, which can be any suitable pump such as an HPLC Pump. Any suitable tubing may be used. In some embodiments, the tubing has a narrow inner diameter, for example, comparable to a syringe needle such as an 8-25 G needle such that sufficient flowability is achieved. Tubing may comprise a slight curvature (or other suitable configuration) to break up the ice as it is dispensed. In some embodiments, the solution is cooled using a heat exchanger 2940 and chiller 2540. As discussed in Bedecarrats et al., 2010, Ice slurry production using super-cooling phenomenon, International Journal of Refrigeration, 33:196-204, the contents of which are incorporated by reference herein, a method of generating ice slurry consists of super-cooling water and subsequently disturbing the water to force crystallization. Dendritic, or branched, growth of the particles, which makes the particles difficult to pump, can be mitigated by using suitable freezing point suppression techniques. This could also be referred to as spontaneous in-solution nucleation.

A controller 2960 controls parameters of the pump 2930, heat exchanger 2940, and chiller 2950, such as flowrate (e.g., 10 ml/min), pressure, and temperature. The controller 2960 communicates with the system components but may be internal to the system or external to the system.

### Scraped Surface Systems

Additional exemplary systems include a scraped surface apparatus, such as a slush ice beverage maker, a super-cooling generator apparatus, a direct refrigerant injection apparatus, such as a direct contact generator, a vacuum triple point of water apparatus, and a crush ice slush generator apparatus. In this method of slurry production, liquid solution is passed through a refrigerated heat exchange which pulls heat away from the liquid. As the liquid freezes to the walls, scrapper blades cyclically remove the ice, breaking it up and allowing it to flow away from the wall. More liquid water is allowed to contact the cold surface, and the cycle repeats until the desired loading of ice is created. This operates using surface nucleation of the crystals. The process may be in-line, continuously producing a slurry composition with the desired characteristics.

Another apparatus utilizes a similar technique as described in U.S. Pat. No. 7,389,653, the contents of which are incorporated herein by reference. Refrigerant is pumped into a lower chamber which is underneath a flexible film. Above the flexible film is another chamber containing water, or a binary solution with water and a freezing point depressant. Heat is pulled out of the top liquid which causes ice to form on the flexible film. An actuator cyclically deforms the film which dislodges the ice. The buoyance of the ice and currents in the liquid created by a whisk element carry the ice away from the film and allow new liquid to repeat the process.

In an embodiment, the system comprises a scraped surface apparatus similar to or the same as an ice cream maker. This method uses nucleation on a very cold surface, which is then scraped using a rotating blade. Small dendritic crystals are created, though the solution must be kept sufficiently viscous to allow the whole mass of the slurry to move as the blade passes round in the container.

In an embodiment, a scraped surface method of slurry production uses a device similar to or the same as a slush ice beverage maker. In a typical commercial slushy machine, a mixture of sugar and water is added to a large mixing tank. Cooling is applied via coils to the bottom of the tank and impellers stir the liquid continuously. As the liquid begins to freeze, large particle growth is prevented due to the high sugar particle concentration which creates spaces between the crystal particles. Also, the continuous motion takes freezing particles away from the coldest area to warmer areas. If sugar levels are too low, their ability to prevent large particle formation diminishes and the risk of block ice forming increases.

Blender and/or Grinder

In one embodiment, a blender architecture of a crushed ice slush generator method may be used. In the blender method, ice particles are broken up into smaller particles by the mechanical force of the blender blade. The blending process may continue while monitoring the particle size distribution until a satisfactory particle size distribution is created.

In an embodiment, a grinder may be used for crushed ice slush generation. As an exemplary grinding method, large ice particles enter a chamber with a continuously tapered grinding surface. The grinding surface tumbles and crushes the particles until the particles are small enough to pass through the thinnest gap between the grinding surface and the inside diameter of the grinding chamber wall. Since the grinder makes the particles smaller and acts as a filter, only particles smaller than the specified size can exit the chamber. As such, the process is controlled and the completion point is predictable. Freezing and jamming of the mechanism may be prevented by continuous motion as well as use of a high torque motor. As an example, a mechanism such as a coffee grinder may be used, as a coffee grinder apparatus allows for setting to a small particle size. For example, the particle size may be set to at or below 100 um. In one embodiment, the system is a static solution nucleation system using a super-cooling generator. A static tank with very smooth walls, filled with liquid that is mixed by an agitator, is provided while being held at a low temperature. The solution will then nucleate and be agitated to limit individual crystal growth within the tank.

In an embodiment, the slurry generation system uses a crushed ice slush generator method of slurry production. The method of slush production utilizes a mechanical impeller to chop up block ice into slurry. The raw materials are added to a blender. The raw materials comprise ice cubes formed from water of a particular size, shape, temperature, and mass, and additives. The blender is turned on for a certain period of time. Once the slush is generated, thermal insulation is used to maintain stability. The slush may be aspirated into a syringe for injection. A peristaltic pump may be used to pump the liquid into a subject.

In one embodiment, a filtration architecture of a crushed ice slush generator method may be used. The filtration architecture may be used to remove large particles from an existing slurry. In this method, a wide distribution of particles are created and then passed through a filtration stage which separates “passing” (small) and “failing” (large) particles. The filter may consist of a woven mesh or a sheet with holes in it. Further, a blender or other mechanical device may be used to break ice into pieces. A refrigeration system may be used to maintain the mixture at a constant temperature.

In one embodiment, a “cheese grater” architecture of a crushed ice slush generator method may be used. In the cheese grater method, an auger or other blade scrapes ice against a perforated sheet which forces only ice chunks smaller than the pore size through. The particles are then collected and used in the slurry composition for administering to a subject. The failing particles continue to recirculate in the auger chamber until they are small enough to exit. For this method, a blender or other mechanical means could be used to break ice into pieces. A refrigeration system could be used to maintain the mixture at a constant temperature.

### Other Systems

In an embodiment, the slurry generation system uses a direct contact generator method of slurry production. A method uses an immiscible primary refrigerant that is evaporated to supersaturate the water and form small smooth crystals. A biocompatible gas may be bubbled through a binary solution. The gas may be stored under pressure as a liquid, and the heat of the phase change may be used to drive flash cooling of the liquid. The violent expansion of the gas may quickly mix the liquid to create small particles.

In an embodiment, the slurry generation system uses a vacuum triple point of water method of slurry production. Spray nozzles are used in this method to generate a mist of particles inside a vacuum chamber. The pressure is controlled in the chamber to encourage freezing of the droplets. Droplet sizes of 50 um were achieved. See Kim, et al., 2001, Study on ice slurry production by water spray, International Journal of Refrigeration, 24(2):176-184, the contents of which are incorporated by reference herein.

In an embodiment, the slurry generation system uses a direct refrigerant injection method of slurry production. This method comprises directly injecting a refrigerant into the slurry liquid as shown in Kiatsiriroat et al., 2000, Ice formation around a jet stream of refrigerant, Energy Conversion & Management, 41:213-221 and Hossain et al., 2004, Ice-slurry production using direct contact heat transfer, International Journal of Refrigeration, 27(5):511-519, the contents of which are incorporated by reference herein. The method creates direct heat transfer between the refrigerant and liquid, thus decreasing thermal resistance between the two. It also creates motion within the liquid which may eliminate the need for other mechanical stirrers.

### Delivery Devices

The slurry generated may be delivered to a subject by any suitable means. In some embodiments, the slurry is injected and the delivery device comprises a cannula such as a needle. Additional examples of delivery devices that may be utilized for injecting the slurry are disclosed in International Application Publication No. PCT/US2017/048995 and U.S. Provisional Application No. 62/381,231, which are incorporated herein by reference in their entirety. Each injection site is the site of a single puncture by, for example, a needle. Treatment of the patient comprises the totality of the injection and deposition sites. Additional approaches to delivery of a slurry utilizing balloon structures are disclosed, for example, in International Application Publication No. PCT/US2018/026273; U.S. Patent Application Publication No. 2018-0289538; and U.S. Provisional Application No. 62/482,008, which are incorporated herein by reference in their entirety.

A system may include a handheld delivery device for administering the slurry by injection. For example, in a continuous flow or hybrid system, the handheld device may be in fluid communication with the circulating system and a continuous flow of the slurry may be received at the handheld device and administered to a subject. This minimizes variation between treatment areas and between individual injections. In another example, the system includes one or more cartridges. The cartridges may be connected to the base station such that the cartridges are in fluid communication with the circulating system. For example, the base station may include one or more ports that the cartridges may be connected to in order to receive a volume of the slurry. In this example, the slurry may be provided at a continuous flow to each of the cartridges, meaning the slurry may circulate throughout the circulation system as well as each of the cartridges simultaneously. Each cartridge may be designed or set to a particular volume, such as 30 mL. In this example, when a cartridge is detached from the port, it contains 30 mL of slurry that is immediately ready to be administered. In some embodiments, the cartridge is designed to deliver up to 2L per injection site. The cartridges may be connected to a handheld device to be administered to a subject through a needle. The handheld device may be driven by a wire drive, a magnetic drive, or the like. Optionally, the handheld device may include a plunger and actuator or a pump such as a peristaltic pump. Also, the handheld device may optionally include coolant in contact with at least a portion of tubing inside the handheld device that contains the slurry. The coolant may be in fluid communication with coolant of the cooling device and be circulated to and from the cooling device via a pump. In the above examples, the continuous flow system decreases treatment time, while increasing effectiveness of the treatment by providing a more consistent slurry.

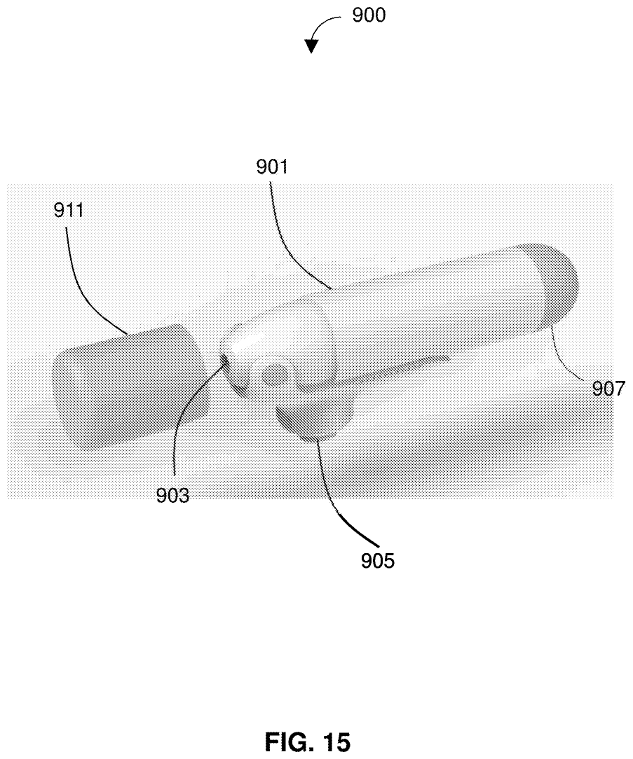

FIG. 15 shows a handheld device 900 for administering a slurry 905. In this embodiment, handheld device 900 includes a body 901, with needle housing 903, adapter 905, end cap 907, and protection cap 911. Handheld device 900 may be used to administer slurry to a subject by injection through a needle connected to needle housing 903. In one example, handheld device 900 may be connected to a tether that is in fluid connection with the circulating system of the system for generating a slurry, at adapter 905. In another example, handheld device 900 may be at least partially filled with slurry and connected to a wire drive, at adapter 905 for dispensing the slurry, as described below. Protection cap 911 may be any suitable cap that is preferably sterile to protect needle housing 903 when it is connected to a needle for administering the slurry and/or when it is not connected to a needle.

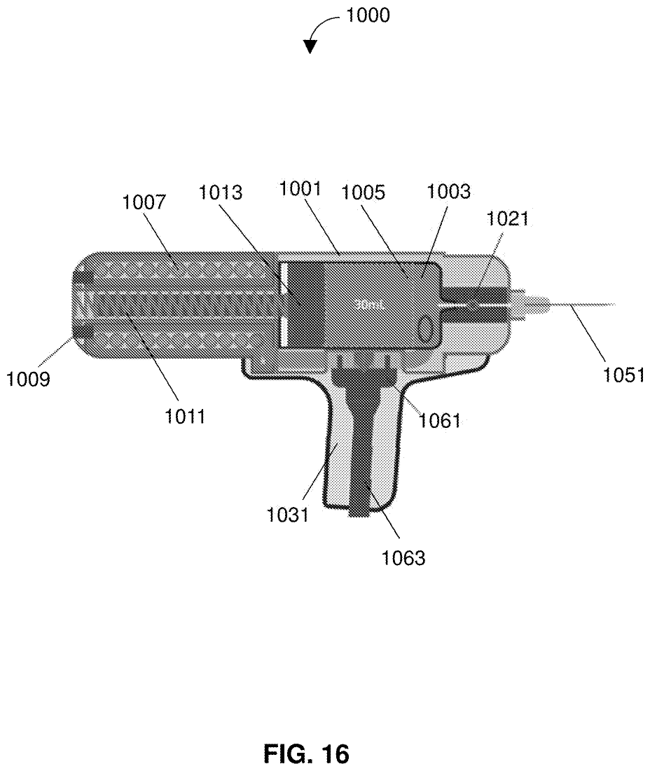

FIG. 16 shows a handheld device 1000 for administering slurry 1005. In this embodiment, handheld device 1000 may be at least partially filled with a volume of slurry 1005 that may be administered to a treatment area of a subject. For example, handheld reservoir 1003 of handheld device 1000 may be at least partially filled with 30 mL of slurry 1005 to be administered to a first treatment area in a subcutaneous region of adipose tissue of the subject. Handheld device 1000 may be used to administer slurry 1005 to a subject through needle 1051, which may be of any suitable gauge size. In some embodiments, the gauge size may be 8-25 G. The handheld device 1000 also includes a plunger 1013 housed within the handheld reservoir 1003, which may be used to determine a volume of injection to a subject, determine a volume of slurry 1005 received by handheld device 1005 when preparing for an injection, and/or to impose a pressure on slurry 1005 in the direction of actuation valve 1021. Plunger 1013 may be controlled by actuator 1011. For example, actuator 1011 may be a mechanically or electro-mechanically controlled spring or other unit able to impose a force on plunger 1013.

As shown, handheld device 1000 includes a body 1001 that may be insulated, tubing 1007 for continuous flow of the slurry 1005 to the system for generating a slurry 1005, the tubing 1007 in fluid communication with at least handheld reservoir 1003. In certain embodiments, tubing 1007 may also be in fluid communication with the circulation system.

In this embodiment, handheld device 1005 further includes peristaltic assembly 1061, which includes a peristaltic pump. Peristaltic assembly 1061 may be connected to wire drive 1063 and a motor (not shown) for driving a peristaltic motion of peristaltic assembly 1061. The peristaltic assembly 1061 may apply a peristaltic motion to tubing 1007 to drive slurry 1005 toward actuation valve 1021 and needle 1051. Actuation valve 1021 may be used to prevent or allow flow of the slurry 1005 to needle 1051 or to modulate a flow of the slurry 1005. For example, the motor may be housed in the base station of the system for generating a slurry 1005. The motor may drive the wire drive 1063 to generate the peristaltic motion, allowing the handheld device 1000 to administer slurry 1005 to a subject.

Handheld device 1000 may further include cooling connection 1009 for handheld device 1000 to receive, remove, or change coolant 1031. As shown, coolant 1031 is in contact with the handheld reservoir 1003, which contains the slurry 1005. Thus, heat may dissipate from handheld reservoir 1003 to coolant 1031 to maintain a temperature of the slurry 1005 while present in handheld device 1000 for injection. In various embodiments, coolant 1031 may be in fluid connection with a coolant of the cooling device and may be circulated to and from the cooling device to the handheld device 1000 via a coolant pump. For example, the coolant 1031 may be the same type of coolant as a coolant contained in the cooling device.

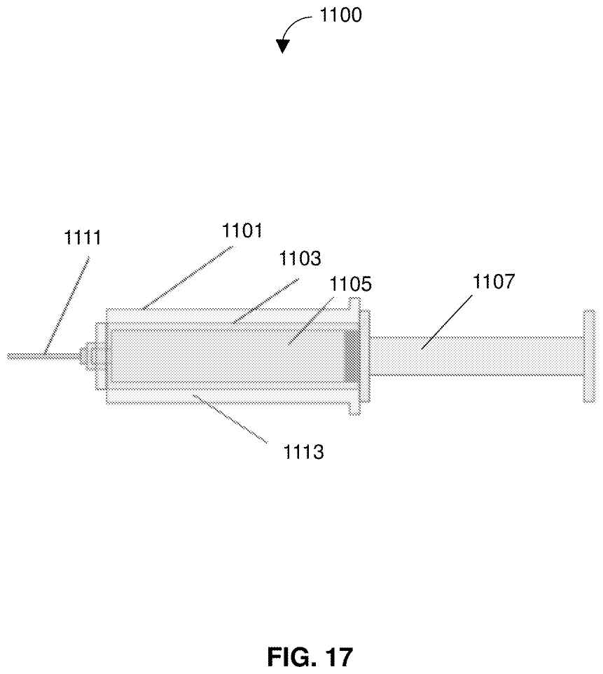

FIG. 17 shows a syringe 1100 for administering slurry 1105. As shown, syringe 1100 includes syringe body 1101, with a volume of slurry 1105 contained within. For example, a treatment volume may include between 10-30 mL of slurry 1105 for a single treatment area, up to 100 mL of slurry 1105, or greater than 100 mL of slurry 1105. Such volume of slurry 1105 may be selected depending on various parameters, such as the intended treatment area, number of intended treatment areas, treatment program or protocol, and characteristic of the subject slurry 1105 is administered to. In embodiments of the present invention up to 2L of slurry may be injected per injection site. Syringe 1100 further includes plunger 1107, which may be depressed to administer slurry 1105. In various embodiments, syringe 1100 may include insulation 1103 at least partially around or in contact with the syringe body 1101. In other embodiments, insulation may be provided as a removable thermal jacket 1113 that is insulated and may be cooled separately in a refrigerator and slid over a part of the syringe body 1101 prior to being filled with the slurry 1105 and/or administration to a subject.

As shown, syringe 1100 is connected to needle 1111, used to administer slurry 1105 to a subject. Needle 1111 may be of any suitable gauge size to administer slurry 1105 by injection. Preferably, the needle has a gauge size that allows for flow of ice particles with a particle size of less than 1 mm. In certain embodiments, the ice particle size is less than 0.25 mm. The gauge size of the needle may be 8 G to 30 G. Preferably, the needle gauge size is chosen based on the patient and treatment and to minimize pain upon injection, minimize the risk of scarring from injection, and minimize the risk of scaring the subject with a large needle size, while being large enough to allow flow of ice particles through the needle.

### Containers

In certain embodiments, systems of the invention comprise containers, for example a cartridge comprising solution to be inserted into a slurry generator described herein. In some embodiments, a system for authenticating containers for use with a slurry generation system is provided. Such a system generally includes a slurry generation device and solution container. The solution container may be a single-use, disposable container that is inserted in the slurry generation device in order to generate a slurry.

The slurry generation device comprises a control system for operating the device, including controlling generation of slurry from the solution container. The slurry generation device further includes a means for authenticating any given container to determine whether the container is suitable and/or authorized to operate with the slurry generation device. In particular, the slurry generation device includes an identification reader for reading data embedded in a container identifier associated with the container upon attachment to or insertion of the container in the slurry generation device. The data from the container identifier, such as an RFID tag, barcode, or chip, is analyzed by the control system and a determination is made as to whether the container is authentic (i.e., suitable for use with the slurry generation device). In the event that the container is determined to be authentic, the control system allows for generation of a slurry using the solution of the container. In the event that the container is determined to not be authentic, the control system prevents generation of a slurry using the solution of the container.

Accordingly, the authentication system ensures that only authorized containers are able to be used with the slurry generation device. The authentication ensures that only those containers recommended and authorized by a manufacturer are to be used, thereby ensuring that the slurry generation system functions as intended and patient safety is maintained.

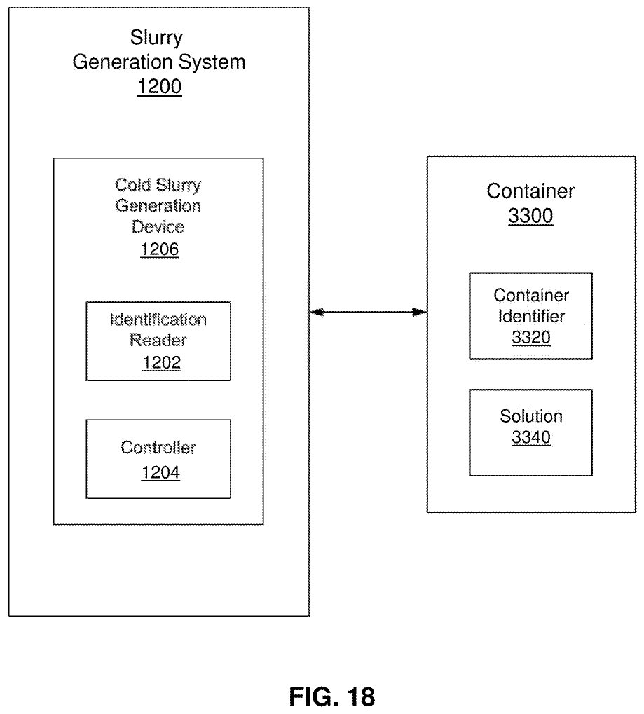

FIG. 18 diagrams a slurry generation system 1200, including a slurry generation device 1206 and container 3300 to be inserted into to the slurry generation device 1206. The slurry generation device 1206 comprises an identification reader 1202 and a controller 1204 (also referred to herein as a “control system 1204”). The container 3300 includes a container identifier 3320 and a solution 3340 and further includes a container housing 3330. Many of the components of the slurry generation system 1200 may be contained in a device housing on a moveable platform or cart 1500, to be provided in a setting in which the procedure is to be performed (e.g., operating room, procedure room, outpatient office setting, etc.) and the container 3300 may be inserted inside the device housing 1207 for use during treatment.

The controller 1204 provides an operator (i.e., surgeon or other medical professional) with control over the generation of a slurry. However, prior to providing an operator with control over generation of the slurry, the container 3300 undergoes an authentication procedure to determine whether the container 3300 is in fact suitable for use with the system 1200. In particular, upon coupling the container 3300 to the system 1200, the identification reader 1202 reads data embedded in the container identifier 3320 of the container 3300, wherein such container identifier data is analyzed to determine authenticity of the container 3300.

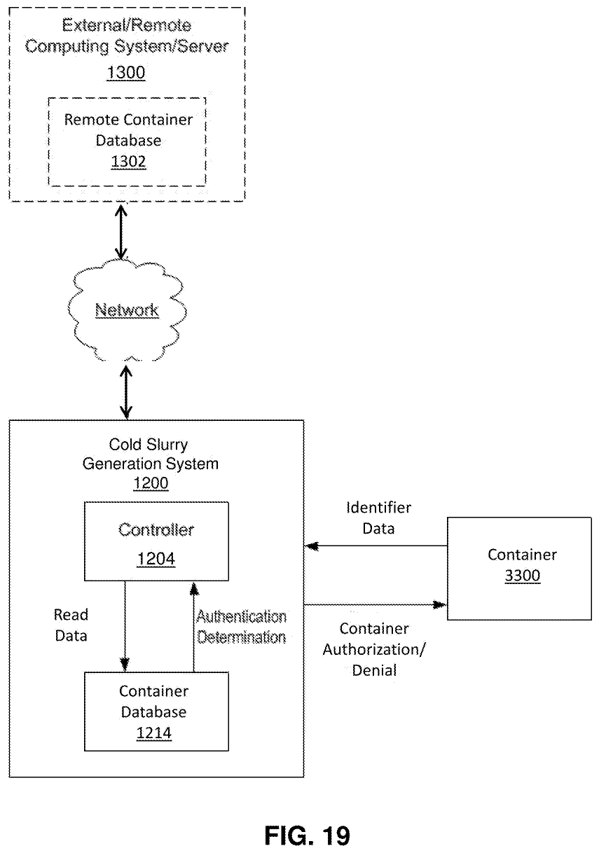

FIG. 19 diagrams the system 1200 and authentication of a container 3300 to be used with the system 1200. The data from the container identifier is read by the identification reader, and then analyzed by the controller 1204. A determination is made as to whether the container is authentic (i.e., suitable for use with the slurry generation device) based on the authentication analysis. In the event that the container is determined to be authentic, the controller 1204 allows for generation of a slurry using the slurry solution of the container 3300 and thus a procedure can be performed using the container 3300. In the event that the container is determined to not be authentic, the controller 1204 prevents generation of a slurry using the solution of the container 3300.

The controller 1204 may include software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. For example, the controller 1204 may include a hardware processor coupled to non-transitory, computer-readable memory containing instructions executable by the processor to cause the controller to carry out various functions of the slurry generation system 1200 as described herein.

The authentication analysis is based on a correlation of the container identifier data with known, predefined authentication data stored in a database, either a local database (i.e., container database 1214) forming part of the system 1200, or a remote database hosted via a remote server 1300 (i.e., remote container database 1302). For example, in some embodiments, the system 1200 may communicate and exchange data with a remote server 1300 over a network. The network may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web).

The known, predefined authentication data stored in the database (database 1214 or database 1302) may be controlled by the owner/manufacturer of the slurry generation device 1206, for example, such that the owner/manufacturer can determine what containers are to be used with the slurry generation device. For example, the owner/manufacturer may set a specific authentication key or provide for specific identity numbers that are proprietary to the owner/manufacturer. As such, the container identifier data for any given container must include a corresponding unique identifier (i.e., authentication key or identity number) in order to be deemed authentic.

One approach to uniquely identifying a container is to authenticate the container by using a private key. In such an approach, both the system 1200 and the container identifier 3320 are taught an identical key. The container identifier may be any suitable container identifier, such as a radio-frequency identification (RFID) tag, a chip, or a barcode. The container identifier 3320 and system 1200 then operate in conjunction to authenticate the key. More specifically, the system 1200 generates a random, unique challenge number. The container identifier 3320 uses this challenge, in combination with the key to generate a response of an authentication code. The method for generating this code (known as a hash function) masks the value of the key. Another approach to uniquely identifying a container is to use unique and unchangeable identity numbers. This approach can be used if there is a region of memory (e.g., a serial or model number), that can only be written by the container identifier manufacturer. The protection is realized by ensuring that the manufacturer only provides container identifiers with legal identification numbers, which prevents simple duplication of legitimate container identifiers.

The container identifier data may include other information and/or characteristics associated with the container. For example, in some embodiments, the container identifier data further includes formulation information of the contents of the solution. In some embodiments, the container identifier data further includes operational history information of the container. As such, in some embodiments, it is further possible to use the controller 1204 to deauthenticate a container based on operational history, such as in the event that the container has already been used, thereby preventing further use of the container.

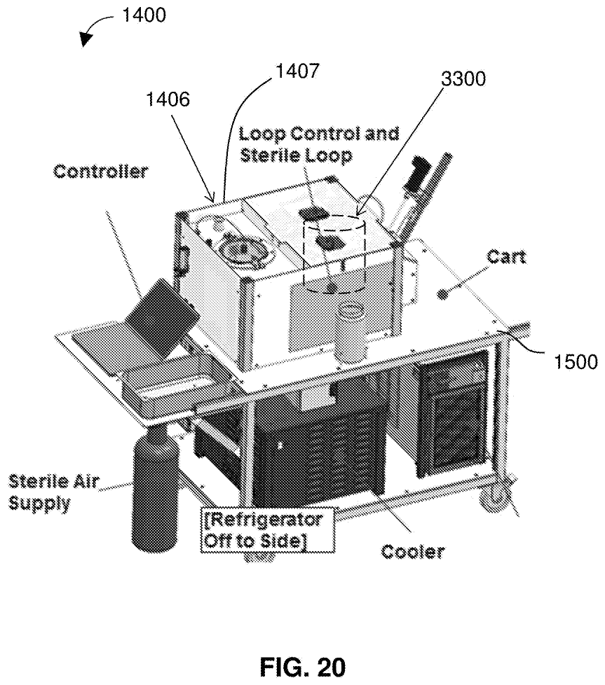

FIG. 20 shows an exemplary embodiment of a slurry generation system 1400. The system includes a controller, such as a laptop, a sterile air supply, a cooler, a refrigerator, a loop control and sterile loop, a cart 1500, slurry generation device 1406, and a container 3300. Components of the system may be contained within a housing 1407. The container 3300 is insertable in the slurry generation device 1406 and contents of the container, namely solution, are used to generate the slurry. Once inserted, the container is in fluid communication with a circulating system in the slurry generation device. The solution is cooled to a certain temperature and ice particles are formed, thereby generating a slurry.

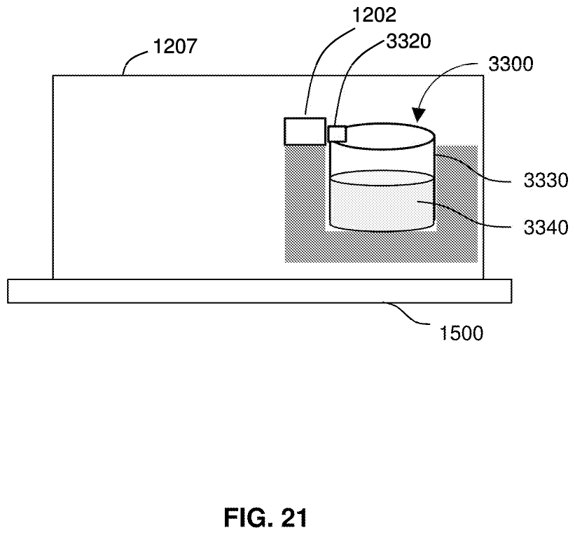

FIG. 21 shows an embodiment of a container 3300 for use with the slurry generation system 1200. The container 3300 can be a single use, disposable unit. The container 3300 generally includes a container housing 3330, solution 3340 disposed within the housing, and a container identifier 3320 disposed on the housing. The container 3300 is configured to be received within a slurry generation device, such as by insertion of the container 3300 in a housing 1207 of the slurry generation device. The container identifier 3320 is provided on the housing 3330 of the container 3300, such that, upon insertion of the container 3300 in the housing 1207 of the slurry generation device, the data embedded in the container identifier 3320 can be read by the identification reader 1202. The housing 3330 of the container may be constructed of any suitable material, such as metal or plastic.

The authentication system ensures that only authorized container are able to be used with the slurry generation device. The containers are authorized to be single-use. If a container is refilled, the system will recognize that the unique container identifier has already been used, thereby preventing use of the container in the slurry generation device. The authentication ensures that only those container recommended and authorized by a manufacturer are to be used, thereby ensuring that the slurry generation system functions as intended and patient safety is maintained.

Analysis conducted in the invention may further include providing a report updating available inventory of containers. For example, the unique container identifier read by the identification reader may be compared to an inventory database in order to remove the unique container from available inventory. The provided report may alert the healthcare provider that container supply is low and additional containers should be ordered.

The authentication further protects against the use of counterfeit components. As counterfeit proprietary components become more prevalent, the need to authenticate original products becomes increasingly necessary. By embedding RFID tags, chips, or barcodes directly into the container and utilizing RFID, chip, or barcode technology for authentication, manufacturers can foil counterfeiters and secure recurring revenue streams, which may otherwise be lost due to counterfeit products.

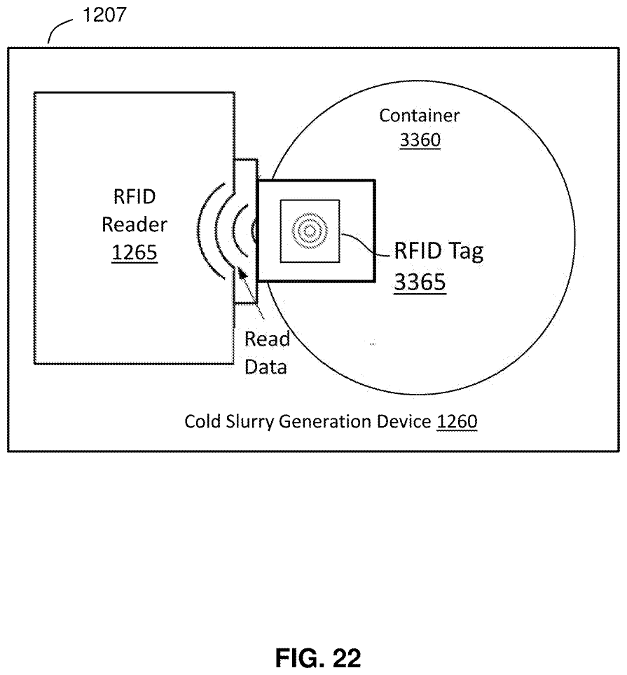

FIG. 22 shows an embodiment of the invention where the identifier is an RFID tag and the identification reader is an RFID reader. As generally understood, RFID technology uses electromagnetic fields to automatically identify and track tags attached to objects. In the invention, the RFID tag associated with the cartridge contains electronically-stored information about the cartridge. The RFID tag may either be read-only, having a factory-assigned serial number that is used as a key into a database, or may be read/write, where object-specific data can be written into the tag by the system user. Field programmable tags may be write-once, read-multiple; “blank” tags may be written with an electronic product code by the user. The RFID tag contains at least three parts: an integrated circuit that stores and processes information and that modulates and demodulates radio-frequency (RF) signals; a means of collecting DC power from the incident reader signal; and an antenna for receiving and transmitting the signal. The tag information is stored in a non-volatile memory. The RFID tag includes either fixed or programmable logic for processing the transmission and sensor data, respectively.

The RFID reader transmits an encoded radio signal to interrogate the tag. The RFID tag receives the message and then responds with its identification and other information. This may be only a unique tag serial number, or may be product-related information such as a stock number, lot or batch number, production date, or other specific information. Since tags have individual serial numbers, the RFID system design can discriminate among several tags that might be within the range of the RFID reader and read them simultaneously. In some embodiments, the RFID tag may be a passive tag, which collects energy from the RFID reader of the system interrogating radio waves. In some embodiments, the RFID tag may be an active tag, which includes a local power source (e.g., a battery) and may operate hundreds of meters from the RFID reader of the system.

FIG. 22 particularly shows the enlarged view of the container 3360 inserted in the slurry generation device 1260 and initial RFID reading, by the RFID reader 1265, to determine authenticity of the container 3360. The data from the chip is analyzed by the controller 1204 and a determination is made as to whether the container is authentic (i.e., suitable for use with the slurry generation device). In the event that the container 3360 is determined to be authentic, the controller allows for generation of a slurry using the solution in the container 3360. In the event that the container 3360 is determined to not be authentic, the controller 1204 prevents generation of a slurry using the solution in the container.

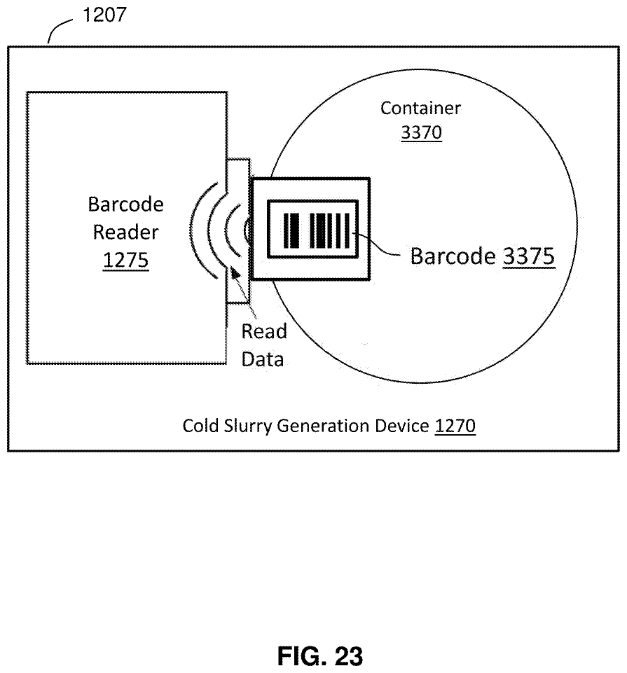

FIG. 23 shows an embodiment of the invention where the identifier is a barcode and the identification reader is a barcode reader. In the invention, the barcode associated with the container contains electronically-stored information about the container. As generally understood, barcode technology uses an optical, machine-readable representation of data. The data describes something about the object that carries the barcode. Traditional barcodes represent data by varying widths and spacing of parallel lines. Two-dimensional (2D) barcodes use rectangles, dots, hexagons, and other geometric patterns. Barcodes may be read or scanned by special optical scanners called barcode readers or other devices or image readers, such as smartphones with cameras that have application software that read images. Traditional barcode scanners are built from a fixed light and a single photosensor that is manually “scrubbed” across the barcode. For example, the RS-232 barcode scanner requires special programming for transferring the input data to the application program, while keyboard interface scanners connect to a computer using an adaptor cable to send the barcode's data to the computer as if it had been typed on the keyboard.

FIG. 23 particularly shows the enlarged view of the container 3370 inserted in the slurry generation device 1270 and initial barcode reading, by the barcode reader 1275, to determine authenticity of the container 3370. The data from the barcode is analyzed by the controller 1204 and a determination is made as to whether the container is authentic (i.e., suitable for use with the slurry generation device). In the event that the container 3370 is determined to be authentic, the controller allows for generation of a slurry using the solution in the container 3370. In the event that the container 3370 is determined to not be authentic, the controller 1204 prevents generation of a slurry using the solution in the container.

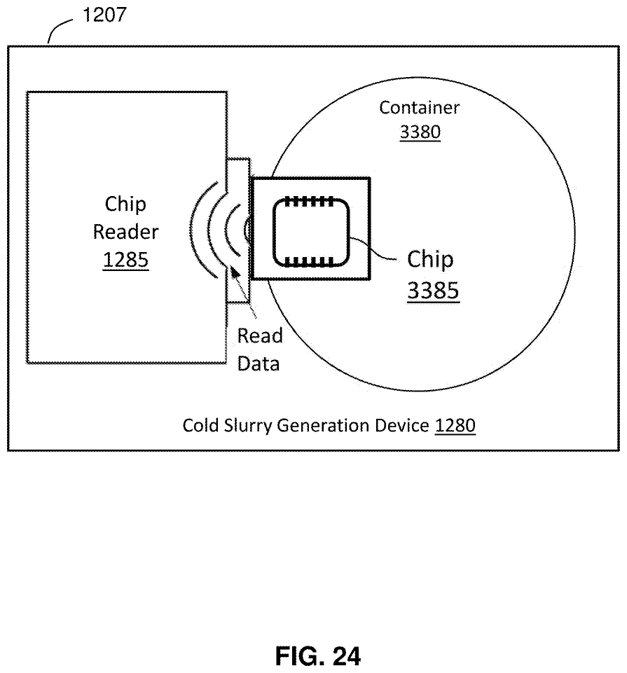

FIG. 24 shows an embodiment of the invention where the identifier is a chip and the identification reader is a chip reader. In the invention, the chip associated with the container contains electronically stored information about the container. As generally understood, chip technology uses integrated circuits, microprocessors, and memory. A chip is a physical electronic authorization device used to control access to a resource and typically has an embedded integrated circuit. Chips can provide identification, authentication, data storage, and application processing. Contactless chips communicate with and are powered by a reader through radio frequency (RF) induction technology and require only proximity to an antenna to communicate. Typically, a chip uses an inductor to capture some of the incident radio-frequency interrogation signal, rectify it, and use it to power the chip's electronics.

FIG. 24 particularly shows the enlarged view of the container 3380 inserted in the slurry generation device 1280 and initial chip reading, by the chip reader 1285, to determine authenticity of the container 3380. The data from the chip is analyzed by the controller 1204 and a determination is made as to whether the container is authentic (i.e., suitable for use with the slurry generation device). In the event that the container 3380 is determined to be authentic, the controller allows for generation of a slurry using the solution in the container 3380. In the event that the container 3380 is determined to not be authentic, the controller 104 prevents generation of a slurry using the solution in the container.

### Solution/Slurry

Systems and methods of the invention are directed to generating a slurry. Solution is provided to one of various slurry generators described herein to generate a slurry. The slurry can be made from any sterile, biocompatible solution that is capable of being cooled to provide a slurry. Sterility is important because the slurry must be safe for injection in human patients.

The solution may comprise water and one or more additives. Additives can be biocompatible ingredients that are safe for use in humans and may include ingredients configured to modify various properties of the solution and/or slurry including the viscosity, freezing point, flowability and tonicity. In some embodiments, additives are inactive ingredients. Any suitable additive may be added to the solution or the slurry in various amounts, including any substance on the FDA GRAS list which is incorporated herein in its entirety.

In some embodiments, the additives comprise one or more of a salt, a sugar, and a thickener. In some embodiments, the salt can comprise saline, potassium, calcium, magnesium, hydrogen phosphate, hydrogen carbonate. In some embodiments, glycerol is an additive. In some embodiments, dextrose is an additive. In some embodiments, an additive may comprise a buffer to stabilize the pH. In some embodiments, the solution pH is about 4.5 to about 9.

The one or more additives may comprise an additive that affects viscosity and/or tonicity. In some embodiments, additives for affecting the viscosity include sodium carboxymethylcellulose (CMC) and Xanthan Gum. In some embodiments, additives for affecting the tonicity include salts, cations, anions, sugars, and sugar alcohols. Tonicity is a characteristic of the solution or slurry related to how it behaves in the subject as a result of its osmolarity/osmolality. Osmolarity is the number of osmoles of solute per volume of solution, measured in Osm/L. Omolality is the number of osmoles of solute per mass of solvent, measured in Osm/kg. A solution or slurry is considered isotonic when it has the same osmolarity as human body fluids creating no osmotic effect, for example water with an osmolarity of 308 mOsm/L does not pass through a cell membrane. A solution or slurry is considered hypotonic when it has a lower osmolarity than human fluids, causing water to pass through the cell membrane into the cell, i.e; the solution or slurry has an osmolarity less than 308 mOsm/L. A solution is hypertonic when it has a higher osmolarity than human fluids, causing water to pass through the cell membrane into the cell, i.e; the solution or slurry has an osmolarity greater than 308 mOsm/L. In certain embodiments, the osmolarity of the slurry is less than about 2,200 milli-Osmoles/kilogram.

The amounts of the additives in the slurry may be within ranges recognized as biocompatible and safe for human use. For example, sodium chloride may be present at about 2.25% by mass or lower in the solution. Glycerol may be present at about 2% by mass or lower in the solution. CMC may be present at about 0.75% by mass or lower in the solution. In an embodiment, the present invention comprises sodium chloride, glycerol, and sodium CMC.

### Nucleation

Methods of the invention control formation of ice particles when generating a slurry by controlling nucleation. Nucleation is the initial process at which ice crystals begin to form, and can be either on a surface, for example a surface of a system component, or in solution. In an agitation system, a continuous flow system and a hybrid system, nucleation occurs in solution. In a scraped surface system and an ice needle system, nucleation occurs at the surface of the system, for example at the surface of a tube. Ice nucleation may be controlled by controlling one or more of the additive content, sterility/particulates in the solution, materials used in the system, parameters of the system, and the inclusion of a nucleator in the system.

Solution additives may affect ice nucleation. Certain additive contents allow for dispersed particulates in the solution, resulting in dispersed formation of ice particles in the slurry. In certain embodiments, additive particulates have a mechanical function and prevent clumping in the system by regulating temperature and agitating the solution. The additives act as mechanical substrates to prevent the additives and/or ice particles from clumping. The additives can be likened to marbles within the solution, regulating the temperature and helping to mix the solution.

The action of ice nucleation is also sensitive to the level of additive particulates in the slurry solution. In some embodiments, methods of the invention include inducing ice nucleation in zones around additive particulates. The additive particulates, although very small, can control the rate of ice slurry nucleation. Nucleation may occur in purified water droplets that are supercooled to −35° C. due to the lack of impurities in the water, whereas water that contains impurities may freeze at −5° C. or warmer. The ice particles contained within the ice slurry are formed via heterogenous nucleation, which can occur between −0° C. and −15° C., depending on when nucleation is triggered during the supercooling process. The impurities in the ice slurry are not necessarily foreign particles introduced in the slurry generation system, but can be components of the solution, for example, CMC. Although water-soluble, CMC at times can precipitate out of solution. When this occurs, the free-floating particles, which become evenly dispersed throughout the ice slurry solution, act as the surface for which heterogenous nucleation is initiated on.

During the process of supercooling, the temperature of the container holding the solution also reduces in temperature. In some embodiments of the invention, the containers are made of plastic, silicone, and metal components. Once nucleation occurs, the components stay at the supercooled temperature, while the slurry itself increases in temperature. If the slurry solution is supercooled too much, issues arise when the slurry interacts with metal components. In some instances, having too low of a temperature causes the occurrence of ice nucleation on the surfaces of the metal components instead of in the slurry solution. This may cause ice particles to stick together on the metal surfaces, thereby causing blocking within the slurry generation and/or slurry injection systems. By controlling initiation of nucleation at a precise temperature, this phenomenon is taken into account in order to ensure slurry nucleation is not hindered by the thermal effects of certain materials in the system. Thus, embodiments of the invention ensure that the slurry does not stick to the metal components of the slurry generation system. In particular, methods of the invention control the supercooling temperature and control the materials and/or material coatings used to ensure slurry does not stick to metal components of the slurry generation system and injectors.

Any defect in the tubing or container results in a cascading effect of increased ice particle formation during nucleation. Thus, using a container and tubing having a smooth interior surface prevents particulates accumulation, thereby preventing the cascading effect. For example, changes to any one of agitation, movement, and flow, may impact another.

Process parameters can be set and/or adjusted to affect nucleation. For example, nucleation requires some degree of cooling prior to initial formation of ice particles. To achieve this, the temperature of the slurry solution can be supercooled within the system. Supercooling is the process of lowering the temperature of the solution below its freezing point. As the solution is cooling down in the slurry generation system, a constant cooling temperature is maintained, and upon the solution reaching a temperature, nucleation can be initiated. In some embodiments, the temperature of the solution is cooled to or below about 10° C., 7° C., 5° C., 4° C., 3° C., 2° C., 1° C., 0° C., −1° C., −2° C., −3° C., −4° C., −5° C., −10° C., −15° C., −20° C., −30° C., −40° C., and −50° C. Temperature control can help in avoiding formation of ice particles on smooth surfaces of the system. In some embodiments, nucleation can be initiated, for example, via a pinch mechanism. In some embodiments, nucleation is spontaneous.

Once nucleation occurs, the ice particles may continue to form in order to reach the target ice coefficient for injection. Ice Coefficient is the percent of ice in the slurry by weight. Slurry generated from the solution can have varying ice coefficients, as provided in International Application No. PCT/US2015/047292, incorporated herein by reference. For example, the slurry can contain between about 2% and about 70% ice by weight.

In certain aspects of the invention, when the target ice coefficient is met, the system shifts into a maintenance mode. Maintenance refers to the process of maintaining the condition of the slurry which includes the ice coefficient and required agitation for ice crystal stability. Maintaining the temperature of the solution provides a slow, controlled formation of ice particles. In some embodiments, the cooling temperature increases to, for example, at or below about 0° C. in maintenance mode and automatically adjusts from that set point to maintain a specific ice coefficient level.

Controlling the formation of ice particles in the slurry allows for control of the size, shape, and amount of ice particles. Characteristics of the ice particles are important, particularly because the slurry must flow through a needle in order to be injected into humans. Therefore, the ice particles must be small enough to fit through the inner diameter of a needle. In some embodiments, ice particles are spherical or globular in shape. Ice particles generated in the invention preferably have dimensions small enough to fit through off-the-shelf needles. In an exemplary embodiment, ice particles are suitable for use with a 14 gauge needle, which has an inner diameter of 1.6 mm. In such an embodiment, the diameter of the ice particles must be smaller than 1.6 mm in order to travel through the needle without causing a blockage.

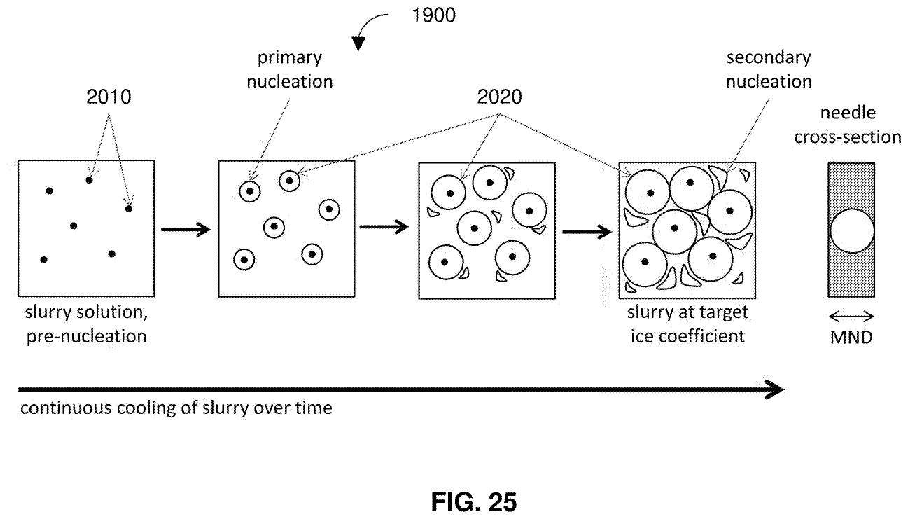

In a solution with fewer seed particles for nucleation, the mechanism by which this maximum ice coefficient is achieved is due to primary nucleation, secondary nucleation, and the continued growth of nucleated crystals. In this scenario, the minimum needle diameter to inject the ice slurry is dependent on the largest ice particle grown (FIG. 25). The act of introducing more seeds to the solution may allow for even more ice particles to be generated upon initiating nucleation, leading to a higher ice coefficient with smaller ice particles that can be expressed through a smaller diameter needle.

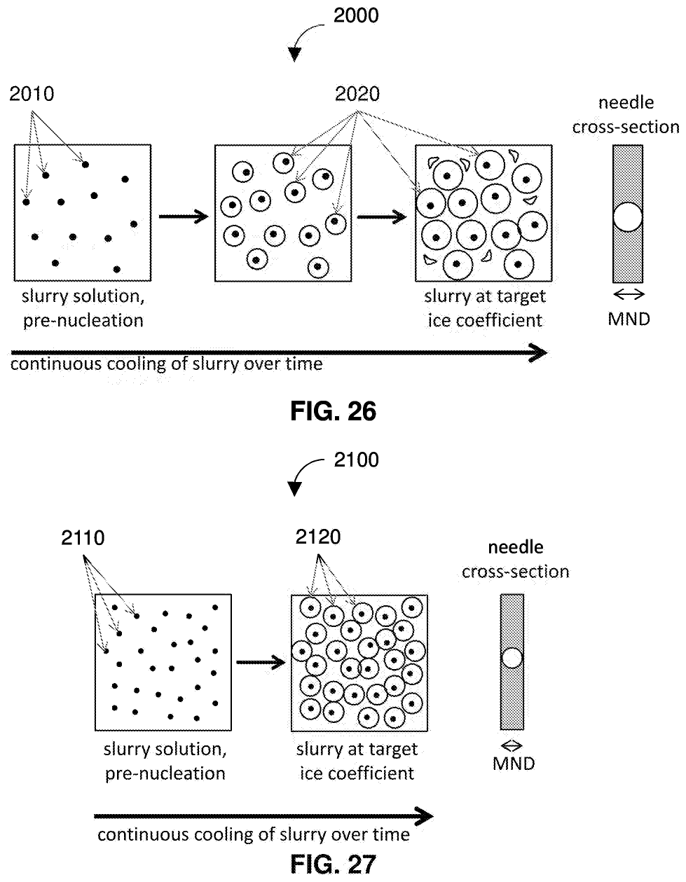

In a solution with more seed particles for nucleation, there is potential to reach the maximum ice coefficient with the largest ice particle grown being smaller than a solution with less seed particles, allowing for slurry to be injected through an even smaller diameter needle (FIG. 26). In a solution with even more seed particles for nucleation, there is potential to reach the maximum ice coefficient immediately after the nucleation event, if there are enough seed particles to attain the maximum ice coefficient. In this scenario, the ice particles could be injected through an even smaller diameter needle due to the ice particles not growing any larger after nucleation (FIG. 27).

FIGS. 25-27 show continuous cooling of slurry over time and formation of ice crystals from solution. The solution is shown pre-nucleation. The slurry at the target ice coefficient and the minimum needle diameter are also shown. Free-floating particles are present in the solution pre-nucleation. The number of free-floating particles varies in FIGS. 25-27. FIG. 25 shows the fewest amount of free-floating particles. When nucleation is initiated, primary nucleation occurs around the free-floating particles to create ice particles. Crystal growth and shearing occurs until the slurry at the target ice coefficient is achieved. Secondary nucleation may occur using the sheared crystals. FIG. 26 shows a moderate amount of free-floating particles. Nucleation is initiated and occurs around the free-floating particles to create ice particles. Crystal growth and shearing occurs until the slurry at the target ice coefficient is achieved, resulting in smaller ice particle size than shown in FIG. 25. FIG. 27 shows the greatest amount of free-floating particles. Nucleation is initiated and occurs around the free-floating particles to create ice particles until the slurry at the target ice coefficient is achieved, resulting in smaller ice particle size than shown in FIG. 26.

In addition to CMC being a seed at which nucleation occurs on, other particles may be placed in the solution in order to establish more points of primary nucleation, in order to generate as many ice particles as possible during the initial nucleation event.

Some potential particles to be used as seeds for heterogenous nucleation include insoluble particles and soluble particles. Insoluble particles include biodegradable polymers, natural polymers, and other options. Biodegradable polymers include Lactide and glycolide polymers, PLGA microspheres and nanoparticles, Poly(lactic acid co-glycolica acid), Polylactic acid, polyglycolic acid, poly(glycolic acid co lactic acid), Caprolactone polymers, Chitosan, Hydroxbutyric acids & hydroxyvaleric acid, Polyanhydrides and polyesters, Poyphosphazenes, Polyphosphoesters, and Lipodisq. Natural polymers include Celluloses (such as carboxymethylcellulose), Carbohydrates and starches (such as amylose& amylopectin), Chitin& Chitosan, Dextrans, Gelatin, Collagen, Elastin, Alginate, Gellan gum, Keratin, Silk, Proteoglycans/glycosaaminoglycans, Lignins, and Polyamino acids.

Soluble particles include freezing point depressants and glycerol, urea, and sugars. Soluble particles also affect osmolarity and tonicity, as discussed in PCT Application Serial Number PCT/US19/54828, the contents of which are incorporated herein by reference in its entirety.

Other options include biologically active molecules, such as synthetic or naturally derived ice nucleating proteins (100-200 kDa) and sterilized, freeze dried bacteria or fractions of bacteria known to be potent ice nucleation activators. Examples of sterilized, freeze dried bacteria or fractions of bacteria, such as Pseudomonas syrinae, Xanthomonas campestris, P. viridiflava, P. fluorescens, and Pantoea agglomerans. Other options also include iron oxides, such as magnetite (see https://www.pnas.org/content/113/43/11986, incorporated herein by reference in its entirety).

### Control Systems

Aspects of the invention described herein, such as monitoring and controlling of various parameters, can be performed using any type of computing device, such as a computer or programmable logic controller (PLC), that includes a processor, e.g., a central processing unit, or any combination of computing devices where each device performs at least part of the process or method. In some embodiments, systems and methods described herein may be performed with a handheld device, e.g., a smart tablet, a smart phone, or a specialty device produced for the system.

Methods of the present disclosure can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. Elements of computer are a processor for executing instructions and one or more memory devices for storing instructions and data. Generally, a computer will also include, or be operatively coupled to receive data from or transfer data to, or both, one or more non-transitory mass storage devices for storing data, e.g., magnetic, magneto-optical disks, or optical disks. In some embodiments, sensors on the system send process data via Bluetooth to a central data collection unit located outside of an incubator. In some embodiments, data is sent directly to the cloud rather than to physical storage devices. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/0 device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to embodiments of the invention involves transforming a tangible, non-transitory, computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines employed in embodiments of the invention may include one or more processors (e.g., a central processing unit (CPU) a graphics processing unit (GPU) or both), a main memory and a static memory, which communicate with each other via a bus.

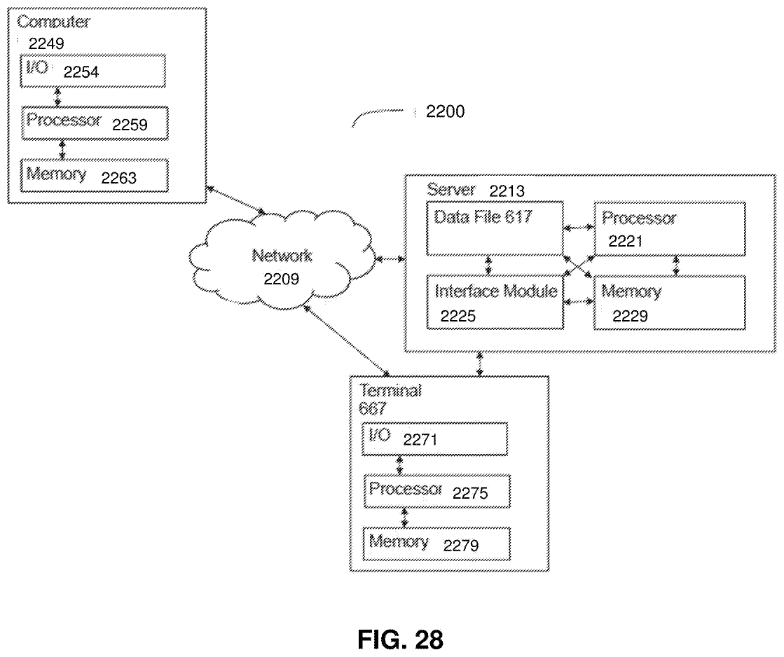

In an example embodiment shown in FIG. 28, system 2200 can include a computer 2249 (e.g., laptop, desktop, or tablet). The computer 2249 may be configured to communicate across a network 2209. Computer 2249 includes one or more processor 2259 and memory 2263 as well as an input/output mechanism 2254. Where methods of the invention employ a client/server architecture, operations of methods of the invention may be performed using server 2213, which includes one or more of processor 2221 and memory 2229, capable of obtaining data, instructions, etc., or providing results via interface module 2225 or providing results as a file 2217. Server 2213 may be engaged over network 2209 through computer 2249 or terminal 2267, or server 2213 may be directly connected to terminal 2267, including one or more processor 2275 and memory 2279, as well as input/output mechanism 2271.

System 2200 or machines according to example embodiments of the invention may further include, for any of I/O 2249, 2237, or 2271 a video display unit (e.g., a liquid crystal display (LCD) or a cathode ray tube (CRT)). Computer systems or machines according to some embodiments can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory 2263, 2279, or 2229 according to example embodiments of the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Systems and methods of the invention use software in certain embodiments to adjust cool-down modes. The solution is cooled down by a cooler, which holds coolant fluid at predetermined temperatures based on software configurations. For example, in a continuous flow or hybrid system, as solution is circulating and cooling down in the slurry generation system, the cooler maintains a constant coolant temperature. Software may be used to garner data surrounding temperature and ice coefficient by using temperature probes placed throughout the exterior of the slurry container, as well as the energy and torque required to maintain a proper rpm for slurry circulation through the system within a circulating peristaltic pump, or from a rotating agitator paddle.

In some embodiments, the system is capable of maintaining slurry after initial readiness for up to one hour, with or without slurry removal from the system. In some embodiments, the system is ready to transfer slurry into syringes within two hours of solution loading.

In certain embodiments, the system has an Off mode, where the system is disconnected from the AC mains. In some embodiments, the system has a Standby mode, which is after power up while waiting for user direction. In some embodiments, the system has an Initialize mode for initial system cooldown prior to loading of solution. In certain embodiments, the system has a Slurry Processing mode for nucleation and maintenance of slurry. In some embodiments, the system has a Transfer mode where the system transfers slurry into a syringe. In certain embodiments, the system has a Planned Shutdown mode to stop active cooling and disable filling of syringes.

In certain embodiments, the slurry generation system is aseptically assembled using terminally sterilized components. Solution to be generated into slurry is transferred into the system via a single-entry port with a male luer fitting that is wiped with Isopropyl alcohol prior to filling. For example, in a hybrid system, the solution enters a sterile loop system and is continually cooled down, circulated, and agitated during slurry generation. Once slurry generation is complete, the slurry may be transferred. In some embodiments, slurry is transferred into an off-the-shelf syringe via the single-entry port. The slurry generation system is effectively a completely closed system, allowing for sterility to be maintained while continually drawing slurry from the system for injection.

Because the slurry is to be injected into humans, several sterility and quality requirements exist for the systems. Sterility may be confirmed by performing any suitable validation tests.

The slurry is deliverable to a subject via injection. The slurry may be delivered by any suitable injection device, such as a cannula, for example, a syringe. The syringe can be formed from any type of biocompatible, pharmacologically inert material suitable for coming in contact with fluids to be provided within a human body. In order to pass through the needle of a syringe without getting stuck or blocking flow of the slurry, the largest cross-section of the ice particles must be smaller than the internal diameter of the needle used for injection.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A system for generating a slurry comprising:
a repository for receiving a solution;
a generator for generating the slurry from the solution; and
a port for transferring the slurry from the system.

2. The system of claim 1, wherein the generator comprises a circulating system comprising a pump in fluid communication with the repository.

3. The system of claim 1, wherein the generator comprises a cooling device for cooling the solution.

4. The system of claim 2, further comprising an agitator.

5. The system of claim 4, wherein the agitator is located in the reservoir.

6. The system of claim 1, wherein the slurry generated is suitable for injection through a cannula.

7. The system of claim 6, wherein the cannula is a needle.

8. The system of claim 7, wherein the needle has a gauge size of about 8 G to about 25 G.

9. The system of claim 1, wherein the solution comprises liquid water and one or more additives.

10. The system of claim 1, wherein the slurry comprises ice particles capable of flowing through a cannula.

11. The system of claim 9, wherein the one or more additives comprise at least one of a salt, a sugar and a thickener.

12. The system of claim 1, wherein the slurry has an ice coefficient of about 2% to about 70%.

13. The system of claim 1, wherein the slurry is configured to be introduced to a patient.

14. The system of claim 13, wherein the slurry is configured to be injected into subcutaneous fat of the patient.

15. The system of claim 1, wherein the slurry has an osmolality of less than about 2,200 milli-Osmoles/kilogram.

16. The system of claim 1, wherein the slurry has a temperature from about −25° C. to about 10° C.

17. The system of claim 1, wherein ice particles in the slurry have a particle size of less than about 1 mm.

18. The system of claim 1, wherein the port is configured for aseptic transfer.

19. The system of claim 1, further comprising a delivery device configured to interlock with the port to fill the delivery device with slurry.

20. The system of claim 19, wherein the delivery device is disposable.

21. The system of claim 20, wherein the delivery device is a cannula.

22. The system of claim 21, wherein the cannula is a needle.

23. The system of claim 20, wherein the delivery device further comprises a thermal jacket.

24. The system of claim 1, further comprising a container configured for insertion in the repository.

25. The system of claim 24, wherein the container is disposable.

26. The system of claim 24, wherein the container comprises pre-mixed solution.

27. The system of claim 24, wherein the container comprises a container identifier selected from the group consisting of a radio-frequency identification (RFID) tag, a chip, or a barcode.

28. A method for generating a slurry comprising:
receiving a solution in a slurry generator; and
generating slurry from the solution, wherein the slurry comprises ice particles capable of flowing through a cannula.

29. The method of claim 28, wherein the method further comprises preparing the solution.

30. The method of claim 29, wherein the solution comprises liquid water and one or more additives.

31. The method of claim 29, wherein preparing the solution further comprises adjusting the one or more additives to generate slurry having ice coefficient, ice particle size, ice shape, ice quality, tonicity, viscosity, pH, and temperature suitable for injection through the cannula.

32. The method of claim 31, wherein the slurry has an ice coefficient of about 2% to about 70%.

33. The method of claim 31, wherein the slurry has an osmolality of less than about 2,200 milli-Osmoles/kilogram.

34. The method of claim 31, wherein the slurry has a temperature from about −25° C. to about 10° C.

35. The method of claim 31, wherein ice particles in the slurry have a particle size of less than about 1 mm.

36. The method of claim 31, wherein the one or more additives comprise at least one of a salt, a sugar and a thickener.

37. The method of claim 28, wherein receiving the solution in the slurry generator comprises inserting a container in a repository of the slurry generator.

38. The method of claim 37, wherein the container is disposable.

39. The method of claim 37, wherein the container comprises pre-mixed solution.

40. The method of claim 37, wherein the container comprises a container identifier selected from the group consisting of a radio-frequency identification (RFID) tag, a chip, or a barcode.

41. The method of claim 28, wherein generating the slurry further comprises generating the slurry in an aseptic system.

42. The method of claim 41, wherein the aseptic system is a closed system.

43. The method of claim 41, wherein generating the slurry comprises cooling and circulating the solution in the slurry generator.

44. The method of claim 41, wherein slurry is generated in the slurry generator when a nucleation event generates ice particles.

45. The method of claim 44, wherein ice nucleation occurs at about 0° C. to about −15° C.

46. The method of claim 45, further comprising switching the system to a maintenance mode when a temperature of the slurry solution reaches at or below about 0° C.

47. The method of claim 46, wherein maintaining the temperature of the solution provides a slow, controlled formation of ice particles.

48. The method of claim 44, wherein inducing ice nucleation further comprises inducing ice nucleation in zones around particulates.

49. The method of claim 28, further comprising preventing accumulation of particulates and unwanted formation of crystals by generating the slurry in a system with smooth surfaces.

50. The method of claim 49, wherein the particulates have a mechanical function and prevent clumping in the system by regulating temperature and agitating the solution.

51. The method of claim 28, wherein the method further comprises aseptically transferring slurry from the slurry generator.

52. The method of claim 51, wherein the aseptic transfer comprises automated aseptic transfer from the slurry generator to a sterile delivery device using a luer connection.

53. The method of claim 52, wherein the delivery device is disposable.

54. The method of claim 52, wherein the delivery device is a handheld device.

55. The method of claim 28, wherein the method further comprises injecting slurry into a subject.

56. The method of claim 55, wherein the slurry is injected into subcutaneous fat of the subject.

57. The method of claim 28, wherein the cannula is a needle.

58. The method of claim 52, wherein the needle has a gauge size of about 8 G to about 25 G.

59. The method of claim 43, further comprising agitating the solution and/or slurry.
